SEQUENCE LISTING (31 sequences)

SEQ ID NO: 1 - DNA, 28 bp, Artificial Sequence
  primer
atcgtccatgnggtnggcnaagntggnt

SEQ ID NO: 2 - DNA, 23 bp, Artificial Sequence
  primer
cggctactagtgcagttcttgaa

SEQ ID NO: 3 - DNA, 29 bp, Artificial Sequence
  primer
atcgtactagtngagnccangtangatnc

SEQ ID NO: 4 - DNA, 24 bp, Artificial Sequence
  primer
cggctactagtttccttcgggcct

SEQ ID NO: 5 - DNA, 23 bp, Artificial Sequence
  primer
atcgtccatggagccagtagatc

SEQ ID NO: 6 - DNA, 24 bp, Artificial Sequence
  primer
atcgtccatgggtggagctatttt

SEQ ID NO: 7 - DNA, 23 bp, Artificial Sequence
  primer
cggctactagtgcgagtttcctt

SEQ ID NO: 8 - DNA, 648 bp, Homo sapiens
atgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtggccaccatcaccatcaccattaa

SEQ ID NO: 9 - protein, 215 aa, Homo sapiens
MGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSGHHHHHH

SEQ ID NO: 10 - DNA, 288 bp, Homo sapiens
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaaactagtggccaccatcaccatcaccattaa

SEQ ID NO: 11 - protein, 95 aa, Homo sapiens
MEPVDPRLEPWKHPGSQPKTACTNCYCKKCCFHCQVCFITKALGISYGRKKRRQRRRPPQGSQTHQVSLSKQPTSQSRGDPTGPKETSGHHHHHH

SEQ ID NO: 12 - DNA, 909 bp, Homo sapiens
atgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtgagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaaactagtggccaccatcaccatcaccattaa

SEQ ID NO: 13 - protein, 302 aa, Homo sapiens
MGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSEPVDPRLEPWKHPGSQPKTACTNCYCKKCCFHCQVCFITKALGISYGRKKRRQRRRPPQGSQTHQVSLSKQPTSQSRGDPTGPKETSGHHHHHH

SEQ ID NO: 14 - DNA, 1029 bp, Homo sapiens
atggatccaaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcttttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtggccaccatcaccatcaccattaa

SEQ ID NO: 15 - protein, 324 aa, Homo sapiens
CSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSGHHHHHH

SEQ ID NO: 16 - DNA, 1290 bp, Homo sapiens
atggatccaaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtgagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaaactagtggccaccatcaccatcaccattaa

SEQ ID NO: 17 - protein, 411 aa, Homo sapiens
CSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSEPVDPRLEPWKHPGSQPKTACTNCYCKKCCFHCQVCFITKALGISYGRKKRRQRRRPPQGSQTHQVSLSKQPTSQSRGDPTGPKETSGHHHHHH

SEQ ID NO: 18 - DNA, 981 bp, Homo sapiens
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtggccaccatcaccatcaccattaa

SEQ ID NO: 19 - protein, 326 aa, Homo sapiens
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSGHHHHHH

SEQ ID NO: 20 - DNA, 1242 bp, Homo sapiens
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtgagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaaactagtggccaccatcaccatcaccattaa

SEQ ID NO: 21 - protein, 413 aa, Homo sapiens
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSEPVDPRLEPWKHPGSQPKTACTNCYCKKCCFHCQVCFITKALGISYGRKKRRQRRRPPQGSQTHQVSLSKQPTSQSRGDPTGPKETSGHHHHHH

SEQ ID NO: 22 - DNA, 288 bp, Homo sapiens
atggagccagtagatcctagactagagccctggaagcatccaggaagtcagcctaaaactgcttgtaccaattgctattgtaaaaagtgttgctttcattgccaagtttgtttcataacagctgccttaggcatctcctatggcaggaagaagcggagacagcgacgaagacctcctcaaggcagtcagactcatcaagtttctctatcaaagcaacccacctcccaatccaaaggggagccgacaggcccgaaggaaactagtggccaccatcaccatcaccattaa

SEQ ID NO: 23 - protein, 95 aa, Homo sapiens
MEPVDPRLEPWKHPGSQPKTACTNCYCKKCCFHCQVCFITAALGISYGRKKRRQRRRPPQGSQTHQVSLSKQPTSQSKGEPTGPKETSGHHHHHH

SEQ ID NO: 24 - DNA, 909 bp, Homo sapiens
atgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcactagtgagccagtagatcctagactagagccctggaagcatccaggaagtcagcctaaaactgcttgtaccaattgctattgtaaaaagtgttgctttcattgccaagtttgtttcataacagctgccttaggcatctcctatggcaggaagaagcggagacagcgacgaagacctcctcaaggcagtcagactcatcaagtttctctatcaaagcaacccacctcccaatccaaaggggagccgacaggcccgaaggaaactagtggccaccatcaccatcaccattaa

SEQ ID NO: 25 - protein, 302 aa, Homo sapiens
MGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCTSEPVDPRLEPWKHPGSQPKTACTNCYCKKCCFHCQVCFITAALGISYGRKKRRQRRRPPQGSQTHQVSLSKQPTSQSKGEPTGPKETSGHHHHHH

SEQ ID NO: 26 - DNA, 57 bp, Homo sapiens
ttcgaaaccatggccgcggactagtggccaccatcaccatcaccattaacggaattc

SEQ ID NO: 27 - protein, 9 aa, Homo sapiens
TSGHHHHHH

SEQ ID NO: 28 - DNA, 58 bp, Homo sapiens
ttcgaaaccatggccgcggactagtggccaccatcaccatcaccattaacgcgaattc

SEQ ID NO: 29 - protein, 9 aa, Homo sapiens
TSGHHHHHH

SEQ ID NO: 30 - DNA, 819 bp, Homo sapiens
atgggtggagctatttccatgaggcggtccaggccgtctggagatctgcgacagagactcttgcgggcgcgtggggagacttatgggagactcttaggagaggtggaagatggatactcgcaatccccaggaggattagacaagggcttgagctcactctcttgtgagggacagaaatacaatcagggacagtatatgaatactccatggagaaacccagctgaagagagagaaaaattagcatacagaaaacaaaatatggatgatatagatgaggaagatgatgacttggtaggggtatcagtgaggccaaaagttcccctaagaacaatgagttacaaattggcaatagacatgtctcattttataaaagaaaaggggggactggaagggatttattacagtgcaagaagacatagaatcttagacatatacttagaaaaggaagaaggcatcataccagattggcaggattacacctcaggaccaggaattagatacccaaagacatttggctggctatggaaattagtccctgtaaatgtatcagatgaggcacaggaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaggttagaagaaggctaaccgcaagaggccttcttaacatggctgacaagaaggaaactcgcactagtggccaccatcaccatcaccattaa

SEQ ID NO: 31 - protein, 272 aa, Homo sapiens
MGGAISMRRSRPSGDLRQRLLRARGETYGRLLGEVEDGYSQSPGGLDKGLSSLSCEGQKYNQGQYMNTPWRNPAEEREKLAYRKQNMDDIDEEDDDLVGVSVRPKVPLRTMSYKLAIDMSHFIKEKGGLEGIYYSARRHRILDIYLEKEEGIIPDWQDYTSGPGIRYPKTFGWLWKLVPVNVSDEAQEDEEHYLMHPAQTSQWDDPWGEVLAWKFDPTLAYTYEAYVRYPEEFGSKSGLSEEEVRRRLTARGLLNMADKKETRTSGHHHHHH

(19) United States
(12) Patent Application Publication
(10) Pub. No.: US 2009/0104229 A1
(43) Pub. Date: Apr. 23, 2009

(54) NOVEL USE

(75) Inventor: Gerald VOSS, Rixensart (BE)

Correspondence Address: GLAXOSMITHKLINE;Corporate Intellectual Property - UW2220, P.O. Box 1539, King of Prussia, PA, 19406-0939, US

(73) Assignee: SmithKline Beecham Biologicals, s.a.

(21) Appl. No.: 12/117,205

(22) Filed: May 8, 2008

Related U.S. Application Data
(62) Division of application No. 11/119,212, filed on Apr. 29, 2005, now abandoned.
Application No. 11/119,212 is a continuation of application No. 10/203,013, filed on Jul. 31, 2002, now abandoned.

(30) Foreign Application Priority Data
Jan. 31, 2000 (GB) 0002200.4
Apr. 14, 2000 (GB) 0009336.9
Jun. 6, 2000 (GB) 0013806.5
Jun. 28, 2000 (EP) PCT/EP00/05998

(51) Int. Cl. A61K 39/00

(52) U.S. Cl. 424/208.1

(57) ABSTRACT

The invention provides the use of a) an HIV Tat protein or polynucleotide; or b) an HIV Nef protein or polynucleotide; or c) an HIV Tat protein or polynucleotide linked to an HIV Nef protein or polynucleotide (Nef-Tat); and an HIV gp120 protein or polynucleotide in the manufacture of a vaccine for the prophylactic or therapeutic immunisation of humans against HIV.

This application is a divisional of U.S. Ser. No. 11/119,212, filed 29 Apr. 2005, which is a continuation of U.S. Ser. No. 10/203,013, filed 31 Jul. 2002, which is a 371 of PCT/EP01/00944, filed 29 Jan. 2001. This application claims benefit of the earlier filing date of PCT/EP00/05998, filed 28 Jun. 2000. The disclosures of each of these applications is incorporated herein by reference. This application also claims benefit of GB application Nos: 0002200.4, filed 31 Jan. 2000; 0009336.9, filed 14 Apr. 2000; and 0013806.5, filed 6 Jun. 2000.

The present invention relates to novel uses of HIV proteins in medicine and vaccine compositions containing such HIV proteins. In particular, the invention relates to the use of HIV Tat and HIV gp120 proteins in combination. Furthermore, the invention relates to the use of HIV Nef and HIV gp120 proteins in combination.

HIV-1 is the primary cause of the acquired immune deficiency syndrome (AIDS) which is regarded as one of the world's major health problems. Although extensive research throughout the world has been conducted to produce a vaccine, such efforts thus far have not been successful.

The HIV envelope glycoprotein gp120 is the viral protein that is used for attachment to the host cell. This attachment is mediated by the binding to two surface molecules of helper T cells and macrophages, known as CD4 and one of the two chemokine receptors CCR-4 or CXCR-5. The gp120 protein is first expressed as a larger precursor molecule (gp160), which is then cleaved post-translationally to yield gp120 and gp41. The gp120 protein is retained on the surface of the virion by linkage to the gp41 molecule, which is inserted into the viral membrane.

The gp120 protein is the principal target of neutralizing antibodies, but unfortunately the most immunogenic regions of the proteins (V3 loop) are also the most variable parts of the protein. Therefore, the use of gp120 (or its precursor gp160) as a vaccine antigen to elicit neutralizing antibodies is thought to be of limited use for a broadly protective vaccine. The gp120 protein does also contain epitopes that are recognized by cytotoxic T lymphocytes (CTL). These effector cells are able to eliminate virus-infected cells, and therefore constitute a second major antiviral immune mechanism. In contrast to the target regions of neutralizing antibodies some CTL epitopes appear to be relatively conserved among different HIV strains. For this reason gp120 and gp160 are considered to be useful antigenic components in vaccines that aim at eliciting cell-mediated immune responses (particularly CTL).

Non-envelope proteins of HIV-1 have been described and include for example internal structural proteins such as the products of the gag and pol genes and, other non-structural proteins such as Rev, Nef, Vif and Tat (Greene et al., New England J. Med, 324, 5, 308 et seq (1991) and Bryant et al. (Ed. Pizzo), Pediatr. Infect. Dis. J., 11, 5, 390 et seq (1992).

HIV Tat and Nef proteins are early proteins, that is, they are expressed early in infection and in the absence of structural protein.

In a conference presentation (C. David Pauza, Immunization with Tat toxoid attenuates SHIV89.6PD infection in rhesus macaques, 12^{th} Cent Gardes meeting, Marnes-La-Coquette, 26.10.1999), experiments were described in which rhesus macaques were immunised with Tat toxoid alone or in combination with an envelope glycoprotein gp160 vaccine combination (one dose recombinant vaccinia virus and one dose recombinant protein). However, the results observed showed that the presence of the envelope glycoprotein gave no advantage over experiments performed with Tat alone.

However, we have found that a Tat- and/or Nef-containing immunogen (especially a Nef-Tat fusion protein) acts synergistically with gp120 in protecting rhesus monkeys from a pathogenic challenge with chimeric human-simian immunodeficiency virus (SHIV). To date the SHIV infection of rhesus macaques is considered to be the most relevant animal model for human AIDS. Therefore, we have used this preclinical model to evaluate the protective efficacy of vaccines containing a gp120 antigen and a Nef- and Tat-containing antigen either alone or in combination. Analysis of two markers of viral infection and pathogenicity, the percentage of CD4-positive cells in the peripheral blood and the concentration of free SHIV RNA genomes in the plasma of the monkeys, indicated that the two antigens acted in synergy. Immunization with either gp120 or NefTat+SIV Nef alone did not result in any difference compared to immunization with an adjuvant alone. In contrast, the administration of the combination of gp120 and NefTat+SIV Nef, antigens resulted in a marked improvement of the two above-mentioned parameters in all animals of those particular experimental group.

Thus, according to the present invention there is provided a new use of HIV Tat and/or Nef protein together with HIV gp120 in the manufacture of a vaccine for the prophylactic or therapeutic immunisation of humans against HIV.

As described above, the NefTat protein, the SIV Nef protein and gp120 protein together give an enhanced response over that which is observed when either NefTat+SIV Nef, or gp120 are used alone. This enhanced response, or synergy can be seen in a decrease in viral load as a result of vaccination with these combined proteins. Alternatively, or additionally the enhanced response manifests itself by a maintenance of CD4+ levels over those levels found in the absence of vaccination with HIV NefTat, SIV Nef and HIV gp120. The synergistic effect is attributed to the combination of gp120 and Tat, or gp120 and Nef, or gp120 and both Nef and Tat.

The addition of other HIV proteins may further enhance the synergistic effect, which was observed between gp120 and Tat and/or Nef. These other proteins may also act synergistically with individual components of the gp120, Tat and/or Nef-containing vaccine, not requiring the presence of the full original antigen combination. The additional proteins may be regulatory proteins of HIV such as Rev, Vif, Vpu, and Vpr. They may also be structural proteins derived from the HIV gag or pol genes.

The HIV gag gene encodes a precursor protein p55, which can assemble spontaneously into immature virus-like particles (VLPs). The precursor is then proteolytically cleaved into the major structural proteins p24 (capsid) and p18 (matrix), and into several smaller proteins. Both the precursor protein p55 and its major derivatives p24 and p18 may be considered as appropriate vaccine antigens which may further enhance the synergistic effect observed between gp120 and Tat and/or Nef. The precursor p55 and the capsid protein p24 may be used as VLPs or as monomeric proteins.

The HIV Tat protein in the vaccine of the present invention may, optionally be linked to an HIV Nef protein, for example as a fusion protein.

The HIV Tat protein, the HIV Nef protein or the NefTat fusion protein in the present invention may have a C terminal Histidine tail which preferably comprises between 5-10 Histidine residues. The presence of an histidine (or ‘His’) tail aids purification.

In a preferred embodiment the proteins are expressed with a Histidine tail comprising between 5 to 10 and preferably six Histidine residues. These are advantageous in aiding purification. Separate expression, in yeast (Saccharomyces cerevisiae), of Nef (Macreadie I. G. et al., 1993, Yeast 9 (6) 565-573) and Tat (Braddock M et al., 1989, Cell 58 (2) 269-79) has been reported. Nef protein and the Gag proteins p55 and p18 are myristilated. The expression of Nef and Tat separately in a Pichia expression system (Nef-His and Tat-His constructs), and the expression of a fusion construct Nef-Tat-His have been described previously in WO99/16884.

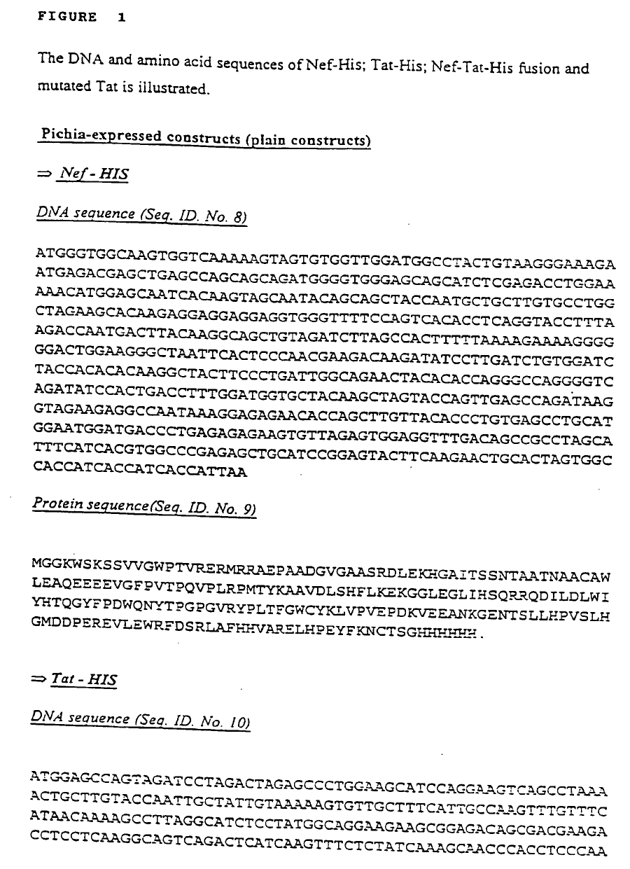

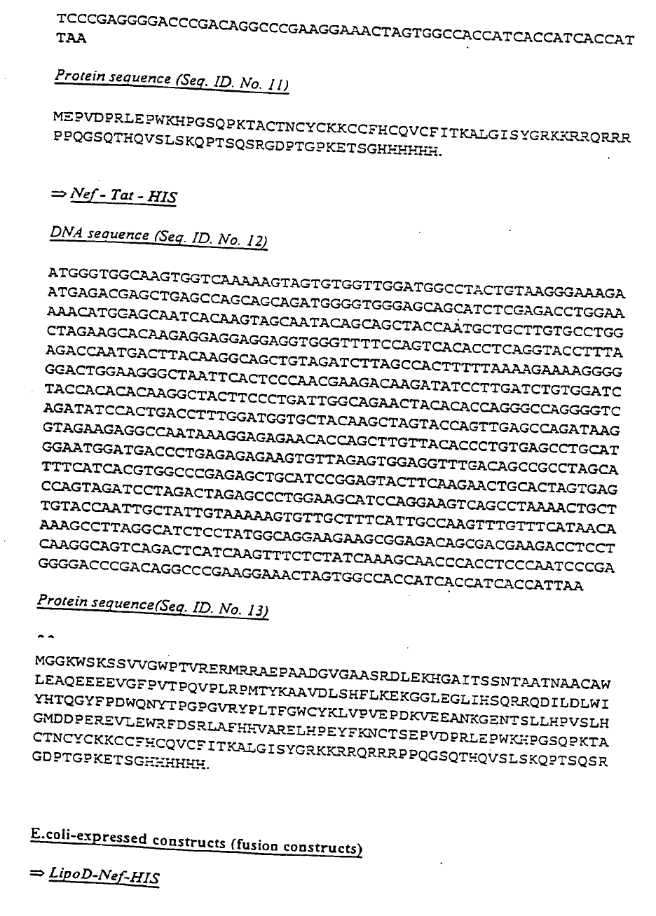

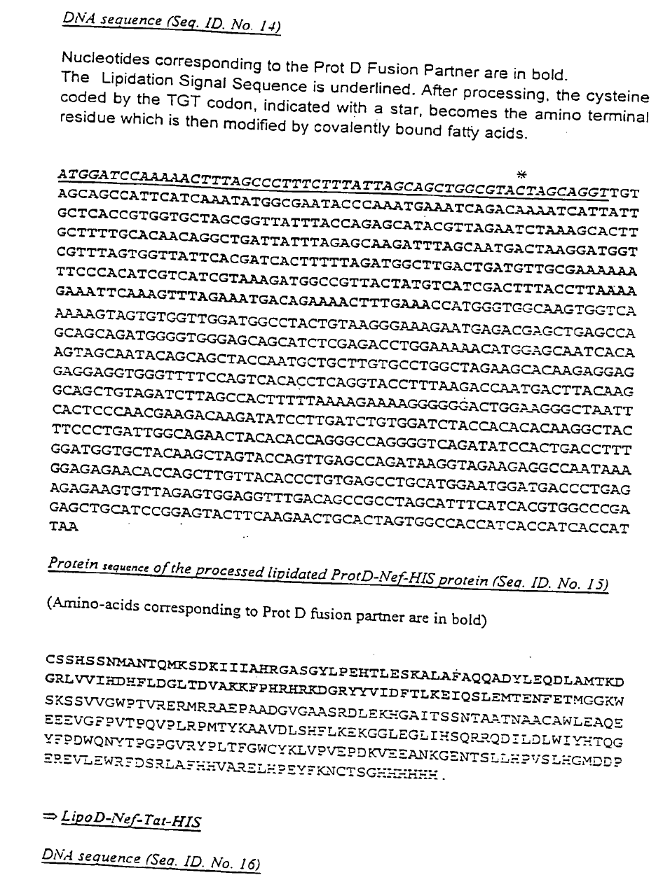

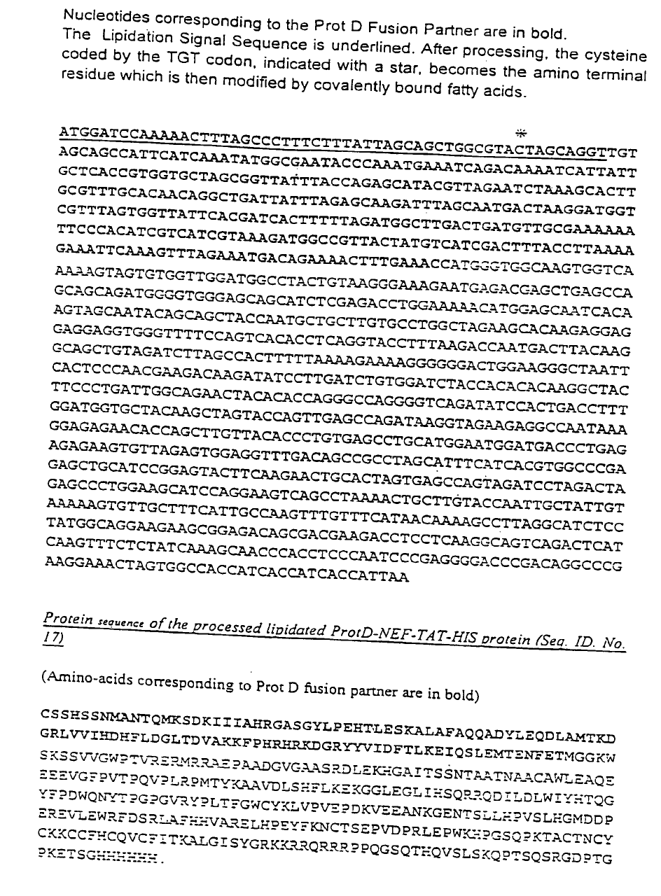

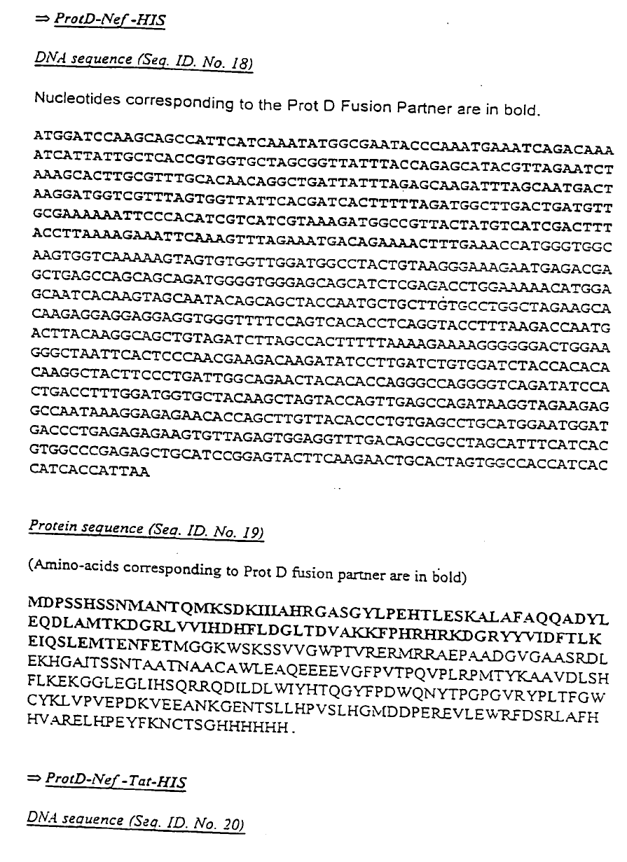

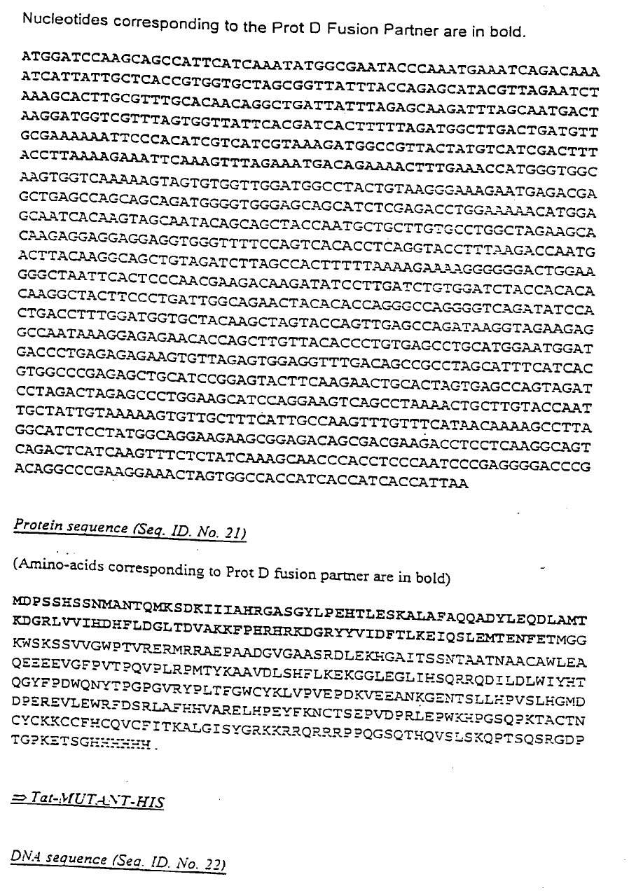

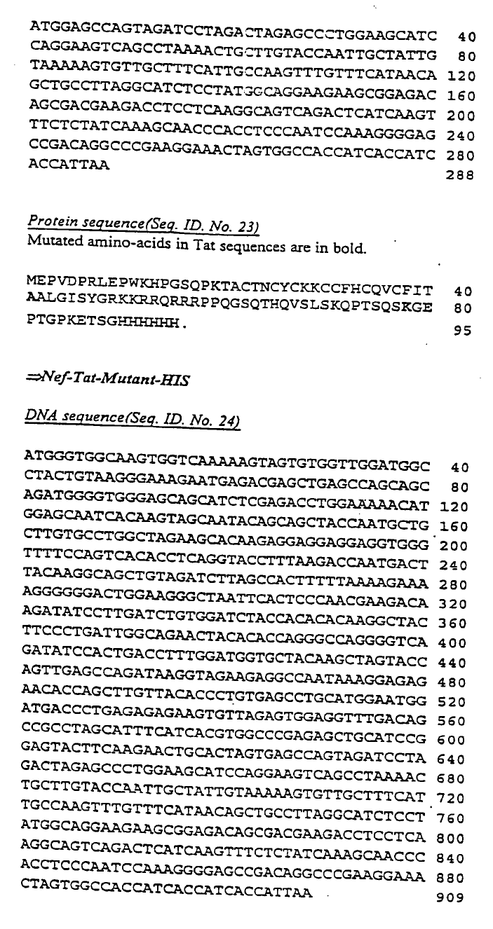

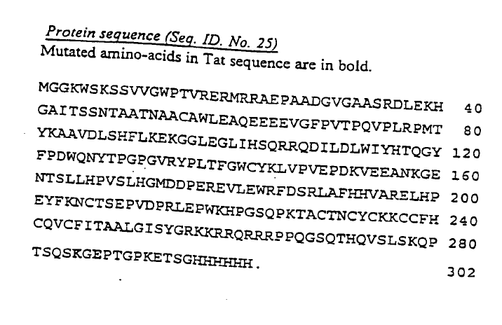

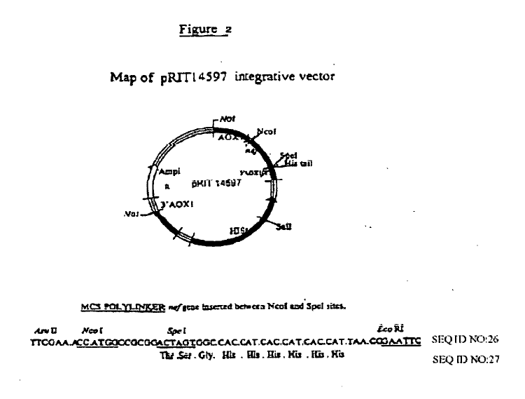

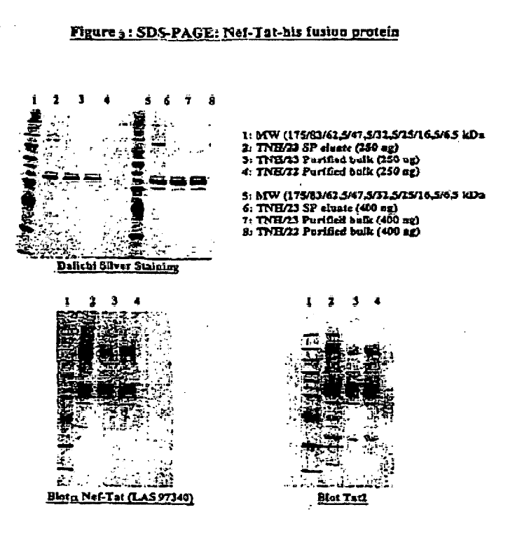

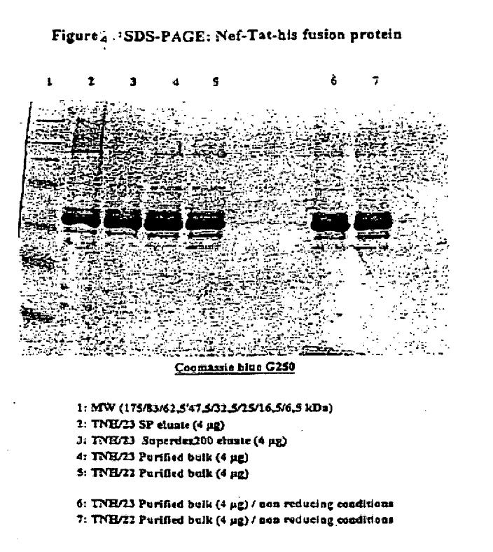

The DNA and amino acid sequences of representative Nef-His (Seq. ID. No.s 8 and 9), Tat-His (Seq. ID. No.s 10 and 11) and of Nef-Tat-His fusion proteins (Seq. ID. No.s 12 and 13) are set forth in FIG. 1.

The HIV proteins of the present invention may be used in their native conformation, or more preferably, may be modified for vaccine use. These modifications may either be required for technical reasons relating to the method of purification, or they may be used to biologically inactivate one or several functional properties of the Tat or Nef protein. Thus the invention encompasses derivatives of HIV proteins which may be, for example mutated proteins. The term ‘mutated’ is used herein to mean a molecule which has undergone deletion, addition or substitution of one or more amino acids using well known techniques for site directed mutagenesis or any other conventional method.

For example, a mutant Tat protein may be mutated so that it is biologically inactive whilst still maintaining its immunogenic epitopes. One possible mutated tat gene, constructed by D. Clements (Tulane University), (originating from BH10 molecular clone) bears mutations in the active site region (Lys41→Ala) and in RGD motif (Arg78→Lys and Asp80→Glu) (Virology 235: 48-64, 1997).

A mutated Tat is illustrated in FIG. 1 (Seq. ID. No.s 22 and 23) as is a Nef-Tat Mutant-His (Seq. ID. No.s 24 and 25).

The HIV Tat or Nef proteins in the vaccine of the present invention may be modified by chemical methods during the purification process to render the proteins stable and monomeric. One method to prevent oxidative aggregation of a protein such as Tat or Nef is the use of chemical modifications of the protein's thiol groups. In a first step the disulphide bridges are reduced by treatment with a reducing agent such as DTT, beta-mercaptoethanol, or gluthatione. In a second step the resulting thiols are blocked by reaction with an alkylating agent (for example, the protein can be carboxyamidated/carbamidomethylated using iodoacetamide). Such chemical modification does not modify functional properties of Tat or Nef as assessed by cell binding assays and inhibition of lymphoproliferation of human peripheral blood mononuclear cells.

The HIV Tat protein and HIV gp120 proteins can be purified by the methods outlined in the attached examples.

The vaccine of the present invention will contain an immunoprotective or immunotherapeutic quantity of the Tat and/or Nef or NefTat and gp120 antigens and may be prepared by conventional techniques.

Vaccine preparation is generally described in New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

The amount of protein in the vaccine dose is selected as an amount which induces an immunoprotective response without significant, adverse side effects in typical vaccinees. Such amount will vary depending upon which specific immunogen is employed. Generally, it is expected that each dose will comprise 1-1000 μg of each protein, preferably 2-200 μg, most preferably 4-40 μg of Tat or Nef or NefTat and preferably 1-150 μg, most preferably 2-25 μg of gp120. An optimal amount for a particular vaccine can be ascertained by standard studies involving observation of antibody titres and other responses in subjects. One particular example of a vaccine dose will comprise 20 μg of NefTat and 5 or 20 μg of gp120. Following an initial vaccination, subjects may receive a boost in about 4 weeks, and a subsequent second booster immunisation.

The proteins of the present invention are preferably adjuvanted in the vaccine formulation of the invention. Adjuvants are described in general in Vaccine Design—the Subunit and Adjuvant Approach, edited by Powell and Newman, Plenum Press, New York, 1995.

Suitable adjuvants include an aluminium salt such as aluminium hydroxide gel (alum) or aluminium phosphate, but may also be a salt of calcium, iron or zinc, or may be an insoluble suspension of acylated tyrosine, or acylated sugars, cationically or anionically derivatised polysaccharides, or polyphosphazenes.

In the formulation of the invention it is preferred that the adjuvant composition induces a preferential Th1 response. However it will be understood that other responses, including other humoral responses, are not excluded.

An immune response is generated to an antigen through the interaction of the antigen with the cells of the immune system. The resultant immune response may be broadly distinguished into two extreme catagories, being humoral or cell mediated immune responses (traditionally characterised by antibody and cellular effector mechanisms of protection respectively). These categories of response have been termed Th1-type responses (cell-mediated response), and Th2-type immune responses (humoral response).

Extreme Th1-type immune responses may be characterised by the generation of antigen specific, haplotype restricted cytotoxic T lymphocytes, and natural killer cell responses. In mice Th1-type responses are often characterised by the generation of antibodies of the IgG2a subtype, whilst in the human these correspond to IgG1 type antibodies. Th2-type immune responses are characterised by the generation of a broad range of immunoglobulin isotypes including in mice IgG1, IgA, and IgM.

It can be considered that the driving force behind the development of these two types of immune responses are cytokines, a number of identified protein messengers which serve to help the cells of the immune system and steer the eventual immune response to either a Th1 or Th2 response. Thus high levels of Th1-type cytokines tend to favour the induction of cell mediated immune responses to the given antigen, whilst high levels of Th2-type cytokines tend to favour the induction of humoral immune responses to the antigen.

It is important to remember that the distinction of Th1 and Th2-type immune responses is not absolute. In reality an individual will support an immune response which is described as being predominantly Th1 or predominantly Th2. However, it is often convenient to consider the families of cytokines in terms of that described in murine CD4+ ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p145-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of IL-4, IL-5, IL-6, IL-10 and tumour necrosis factor-β(TNF-β).

It is known that certain vaccine adjuvants are particularly suited to the stimulation of either Th1 or Th2-type cytokine responses. Traditionally the best indicators of the Th1:Th2 balance of the immune response after a vaccination or infection includes direct measurement of the production of Th1 or Th2 cytokines by T lymphocytes in vitro after restimulation with antigen, and/or the measurement of the IgG1:IgG2a ratio of antigen specific antibody responses.

Thus, a Th1-type adjuvant is one which stimulates isolated T-cell populations to produce high levels of Th1-type cytokines when re-stimulated with antigen in vitro, and induces antigen specific immunoglobulin responses associated with Th1-type isotype.

Preferred Th1-type immunostimulants which may be formulated to produce adjuvants suitable for use in the present invention include and are not restricted to the following.

Monophosphoryl lipid A, in particular 3-de-O-acylated monophosphoryl lipid A (3D-MPL), is a preferred Th1-type immunostimulant for use in the invention. 3D-MPL is a well known adjuvant manufactured by Ribi Immunochem, Montana. Chemically it is often supplied as a mixture of 3-de-O-acylated monophosphoryl lipid A with either 4, 5, or 6 acylated chains. It can be purified and prepared by the methods taught in GB 2122204B, which reference also discloses the preparation of diphosphoryl lipid A, and 3-O-deacylated variants thereof. Other purified and synthetic lipopolysaccharides have been described (U.S. Pat. No. 6,005,099 and EP 0 729 473 B1; Hilgers et al., 1986, Int. Arch. Allergy. Immunol., 79(4):392-6; Hilgers et al., 1987, Immunology, 60(1):141-6; and EP 0 549 074 B1). A preferred form of 3D-MPL is in the form of a particulate formulation having a small particle size less than 0.2 μm in diameter, and its method of manufacture is disclosed in EP 0 689 454.

Saponins are also preferred Th1 immunostimulants in accordance with the invention. Saponins are well known adjuvants and are taught in: Lacaille-Dubois, M and Wagner H. (1996. A review of the biological and pharmacological activities of saponins. Phytomedicine vol 2 pp 363-386). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit. Rev Ther Drug Carrier Syst, 1996, 12 (1-2):1-55; and EP 0 362 279 B1. The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Also described in these references is the use of QS7 (a non-haemolytic fraction of Quil-A) which acts as a potent adjuvant for systemic vaccines. Use of QS21 is further described in Kensil et al. (1991. J. Immunology vol 146, 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711.

Another preferred immunostimulant is an immunostimulatory oligonucleotide containing unmethylated CpG dinucleotides (“CpG”). CpG is an abbreviation for cytosine-guanosine dinucleotide motifs present in DNA. CpG is known in the art as being an adjuvant when administered by both systemic and mucosal routes (WO 96/02555, EP 468520, Davis et al., J. Immunol, 1998, 160(2):870-876; McCluskie and Davis, J. Immunol., 1998, 161(9):4463-6). Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the CG motif is not methylated, but other unmethylated CpG sequences are known to be immunostimulatory and may be used in the present invention.

In certain combinations of the six nucleotides a palindromic sequence is present. Several of these motifs, either as repeats of one motif or a combination of different motifs, can be present in the same oligonucleotide. The presence of one or more of these immunostimulatory sequences containing oligonucleotides can activate various immune subsets, including natural killer cells (which produce interferon γ and have cytolytic activity) and macrophages (Wooldrige et al Vol 89 (no. 8), 1977). Other unmethylated CpG containing sequences not having this consensus sequence have also now been shown to be immunomodulatory.

CpG when formulated into vaccines, is generally administered in free solution together with free antigen (WO 96/02555; McCluskie and Davis, supra) or covalently conjugated to an antigen (WO 98/16247), or formulated with a carrier such as aluminium hydroxide ((Hepatitis surface antigen) Davis et al. supra; Brazolot-Millan et al., Proc. Natl. Acad. Sci., USA, 1998, 95(26), 15553-8).

Such immunostimulants as described above may be formulated together with carriers, such as for example liposomes, oil in water emulsions, and or metallic salts, including aluminium salts (such as aluminium hydroxide). For example, 3D-MPL may be formulated with aluminium hydroxide (EP 0 689 454) or oil in water emulsions (WO 95/17210); QS21 may be advantageously formulated with cholesterol containing liposomes (WO 96/33739), oil in water emulsions (WO 95/17210) or alum (WO 98/15287); CpG may be formulated with alum (Davis et al. supra; Brazolot-Millan supra) or with other cationic carriers.

Combinations of immunostimulants are also preferred, in particular a combination of a monophosphoryl lipid A and a saponin derivative (WO 94/00153; WO 95/17210; WO 96/33739; WO 98/56414; WO 99/12565; WO 99/11241), more particularly the combination of QS21 and 3D-MPL as disclosed in WO 94/00153. Alternatively, a combination of CpG plus a saponin such as QS21 also forms a potent adjuvant for use in the present invention.

Thus, suitable adjuvant systems include, for example, a combination of monophosphoryl lipid A, preferably 3D-MPL, together with an aluminium salt. An enhanced system involves the combination of a monophosphoryl lipid A and a saponin derivative particularly the combination of QS21 and 3D-MPL as disclosed in WO 94/00153, or a less reactogenic composition where the QS21 is quenched in cholesterol containing liposomes (DQ) as disclosed in WO 96/33739.

A particularly potent adjuvant formulation involving QS21, 3D-MPL & tocopherol in an oil in water emulsion is described in WO 95/17210 and is another preferred formulation for use in the invention.

Another preferred formulation comprises a CpG oligonucleotide alone or together with an aluminium salt.

In another aspect of the invention, the vaccine may contain DNA encoding one or more of the Tat, Nef and gp120 polypeptides, such that the polypeptide is generated in situ. The DNA may be present within any of a variety of delivery systems known to those of ordinary skill in the art, including nucleic acid expression systems such as plasmid DNA, bacteria and viral expression systems. Numerous gene delivery techniques are well known in the art, such as those described by Rolland, Crit. Rev. Therap. Drug Carrier Systems 15:143-198, 1998 and references cited therein. Appropriate nucleic acid expression systems contain the necessary DNA sequences for expression in the patient (such as a suitable promoter and terminating signal). When the expression system is a recombinant live microorganism, such as a virus or bacterium, the gene of interest can be inserted into the genome of a live recombinant virus or bacterium. Inoculation and in vivo infection with this live vector will lead to in vivo expression of the antigen and induction of immune responses. Viruses and bacteria used for this purpose are for instance: poxviruses (e.g; vaccinia, fowlpox, canarypox, modified poxviruses e.g. Modified Virus Ankara (MVA)), alphaviruses (Sindbis virus, Semliki Forest Virus, Venezuelian Equine Encephalitis Virus), flaviviruses (yellow fever virus, Dengue virus, Japanese encephalitis virus), adenoviruses, adeno-associated virus, picornaviruses (poliovirus, rhinovirus), herpesviruses (varicella zoster virus, etc), Listeria, Salmonella, Shigella, Neisseria, BCG. These viruses and bacteria can be virulent, or attenuated in various ways in order to obtain live vaccines. Such live vaccines also form part of the invention.

Thus, the Nef, Tat and gp120 components of a preferred vaccine according to the invention may be provided in the form of polynucleotides encoding the desired proteins.

Furthermore, immunisations according to the invention may be performed with a combination of protein and DNA-based formulations. Prime-boost immunisations are considered to be effective in inducing broad immune responses. Adjuvanted protein vaccines induce mainly antibodies and T helper immune responses, while delivery of DNA as a plasmid or a live vector induces strong cytotoxic T lymphocyte (CTL) responses. Thus, the combination of protein and DNA vaccination will provide for a wide variety of immune responses. This is particularly relevant in the context of HIV, since both neutralising antibodies and CTL are thought to be important for the immune defence against HIV.

In accordance with the invention a schedule for vaccination with gp120, Nef and Tat, alone or in combination, may comprise the sequential (“prime-boost”) or simultaneous administration of protein antigens and DNA encoding the above-mentioned proteins. The DNA may be delivered as plasmid DNA or in the form of a recombinant live vector, e.g. a poxvirus vector or any other suitable live vector such as those described herein. Protein antigens may be injected once or several times followed by one or more DNA administrations, or DNA may be used first for one or more administrations followed by one or more protein immunisations.

A particular example of prime-boost immunisation according to the invention involves priming with DNA in the form of a recombinant live vector such as a modified poxvirus vector, for example Modified Virus Ankara (MVA) or a alphavirus, for example Venezuelian Equine Encephalitis Virus followed by boosting with a protein, preferably an adjuvanted protein.

Thus the invention further provides a pharmaceutical kit comprising:
a) a composition comprising one or more of gp120, Nef and Tat proteins together with a pharmaceutically acceptable excipient; and
b) a composition comprising one or more of gp120, Nef and Tat-encoding polynucleotides together with a pharmaceutically acceptable excipient;
   with the proviso that at least one of (a) or (b) comprises gp120 with Nef and/or Tat and/or Nef-Tat.

Compositions a) and b) may be administered separately, in any order, or together. Preferably a) comprises all three of gp120, Nef and Tat proteins. Preferably b) comprises all three of gp120, Nef and Tat DNA. Most preferably the Nef and Tat are in the form of a NefTat fusion protein.

In a further aspect of the present invention there is provided a method of manufacture of a vaccine formulation as herein described, wherein the method comprises admixing a combination of proteins according to the invention. The protein composition may be mixed with a suitable adjuvant and, optionally, a carrier.

Particularly preferred adjuvant and/or carrier combinations for use in the formulations according to the invention are as follows:

### i) 3D-MPL+QS21 in DQ

### ii) Alum+3D-MPL

iii) Alum+QS21 in DQ+3D-MPL

### iv) Alum+CpG

v) 3D-MPL+QS21 in DQ+oil in water emulsion

### vi) CpG

The invention is illustrated in the accompanying examples and Figures:

### EXAMPLES

### General

The Nef gene from the Bru/Lai isolate (Cell 40: 9-17, 1985) was selected for the constructs of these experiments since this gene is among those that are most closely related to the consensus Nef.

The starting material for the Bru/Lai Nef gene was a 1170 bp DNA fragment cloned on the mammalian expression vector pcDNA3 (pcDNA3/Nef).

The Tat gene originates from the BH10 molecular clone. This gene was received as an HTLV III cDNA clone named pCV1 and described in Science, 229, p69-73, 1985.

The expression of the Nef and Tat genes could be in Pichia or any other host.

### Example 1

### Expression of HIV-1 Nef and Tat Sequences in Pichia pastoris

Nef protein, Tat protein and the fusion Nef-Tat were expressed in the methylotrophic yeast Pichia pastoris under the control of the inducible alcohol oxidase (AOX1) promoter.

To express these HIV-1 genes a modified version of the integrative vector PHIL-D2 (INVITROGEN) was used. This vector was modified in such a way that expression of heterologous protein starts immediately after the native ATG codon of the AOX1 gene and will produce recombinant protein with a tail of one glycine and six histidines residues. This PHIL-D2-MOD vector was constructed by cloning an oligonucleotide linker between the adjacent AsuII and EcoRI sites of PHIL-D2 vector (see FIG. 2). In addition to the His tail, this linker carries NcoI, SpeI and XbaI restriction sites between which nef, tat and nef-tat fusion were inserted.

1.1 Construction of the Integrative Vectors pRIT14597 (Encoding Nef-His Protein), pRIT14598 (Encoding Tat-His Protein) and pRIT14599 (encoding Fusion Nef-Tat-His).

The nef gene was amplified by PCR from the pcDNA3/Nef plasmid with primers 01 and 02.

```
PRIMER 01 (Seq ID NO 1):
              NcoI
5′ATCGTCCATG.GGT.GGC.AAG.TGG.T 3′
PRIMER 02 (Seq ID NO 2):
              SpeI
5′CGGCTACTAGTGCAGTTCTTGAA 3′
```

The PCR fragment obtained and the integrative PHIL-D2-MOD vector were both restricted by NcoI and SpeI, purified on agarose gel and ligated to create the integrative plasmid pRIT14597 (see FIG. 2).

The tat gene was amplified by PCR from a derivative of the pCV1 plasmid with primers 05 and 04:

```
PRIMER 04 (Seq ID NO 4):
              SpeI
5′CGGCTACTAGTTTCCTTCGGGCCT 3′
PRIMER 05 (Seq ID NO 5):
              NcoI
5′ATCGTCCATGGAGCCAGTAGATC 3′
```

An NcoI restriction site was introduced at the 5′ end of the PCR fragment while a SpeI site was introduced at the 3′ end with primer 04. The PCR fragment obtained and the PHIL-D2-MOD vector were both restricted by NcoI and SpeI, purified on agarose gel and ligated to create the integrative plasmid pRIT14598.

To construct pRIT14599, a 910 bp DNA fragment corresponding to the nef-tat-His coding sequence was ligated between the EcoRI blunted(T4 polymerase) and NcoI sites of the PHIL-D2-MOD vector. The nef-tat-His coding fragment was obtained by XbaI blunted(T4 polymerase) and NcoI digestions of pRIT 14596.

1.2 Transformation of Pichia pastoris Strain GS115(his4).

To obtain Pichia pastoris strains expressing Nef-His, Tat-His and the fusion Nef-Tat-His, strain GS115 was transformed with linear NotI fragments carrying the respective expression cassettes plus the HIS4 gene to complement his4 in the host genome. Transformation of GS115 with NotI-linear fragments favors recombination at the AOXI locus.

Multicopy integrant clones were selected by quantitative dot blot analysis and the type of integration, insertion (Mut⁺ phenotype) or transplacement (Mut^{s}phenotype), was determined.

From each transformation, one transformant showing a high production level for the recombinant protein was selected:

Strain Y1738 (Mut⁺ phenotype) producing the recombinant Nef-His protein, a myristylated 215 amino acids protein which is composed of:
Myristic acid
A methionine, created by the use of NcoI cloning site of PHIL-D2-MOD vector
205 a.a. of Nef protein (starting at a.a.2 and extending to a.a.206)
A threonine and a serine created by the cloning procedure (cloning at SpeI site of PHIL-D2-MOD vector.
One glycine and six histidines.

Strain Y1739 (Mut⁺ phenotype) producing the Tat-His protein, a 95 amino acid protein which is composed of:
A methionine created by the use of NcoI cloning site
85 a.a. of the Tat protein (starting at a.a.2 and extending to a.a.86)
A threonine and a serine introduced by cloning procedure
One glycine and six histidines

Strain Y1737(Mut^{s} phenotype) producing the recombinant Nef-Tat-His fusion protein, a myristylated 302 amino acids protein which is composed of:
Myristic acid
A methionine, created by the use of NcoI cloning site
205a.a. of Nef protein (starting at a.a.2 and extending to a.a.206)
A threonine and a serine created by the cloning procedure
85a.a. of the Tat protein (starting at a.a.2 and extending to a.a.86)
A threonine and a serine introduced by the cloning procedure
One glycine and six histidines

### Example 2

### Expression of HIV-1 Tat-Mutant in Pichia pastoris

A mutant recombinant Tat protein has also been expressed. The mutant Tat protein must be biologically inactive while maintaining its immunogenic epitopes.

A double mutant tat gene, constructed by D. Clements (Tulane University) was selected for these constructs.

This tat gene (originates from BH10 molecular clone) bears mutations in the active site region (Lys41→Ala) and in RGD motif (Arg78→Lys and Asp80→Glu) (Virology 235: 48-64, 1997).

The mutant tat gene was received as a cDNA fragment subcloned between the EcoRI and HindIII sites within a CMV expression plasmid (pCMVLys41/KGE)

### 2.1 Construction of the Integrative Vectors

pRIT14912(Encoding Tat Mutant-His Protein) and pRIT14913(Encoding Fusion Nef-Tat Mutant-His).

The tat mutant gene was amplified by PCR from the pCMVLys41/KGE plasmid with primers 05 and 04 (see section 1.1construction of pRIT14598)

An NcoI restriction site was introduced at the 5′ end of the PCR fragment while a SpeI site was introduced at the 3′ end with primer 04. The PCR fragment obtained and the PHIL-D2-MOD vector were both restricted by NcoI and SpeI, purified on agarose gel and ligated to create the integrative plasmid pRIT14912

To construct pRIT14913, the tat mutant gene was amplified by PCR from the pCMVLys41/KGE plasmid with primers 03 and 04.

```
PRIMER 03 (Seq ID NO 3):
              SpeI
5′ATCGTACTAGT.GAG.CCA.GTA.GAT.C 3′
PRIMER 04 (Seq ID NO 4):
              SpeI
5′CGGCTACTAGTTTCCTTCGGGCCT 3′
```

The PCR fragment obtained and the plasmid pRIT14597 (expressing Nef-His protein) were both digested by SpeI restriction enzyme, purified on agarose gel and ligated to create the integrative plasmid pRIT14913

2.2 Transformation of Pichia pastoris Strain GS115.

Pichia pastoris strains expressing Tat mutant-His protein and the fusion Nef-Tat mutant-His were obtained, by applying integration and recombinant strain selection strategies previously described in section 1.2.

Two recombinant strains producing Tat mutant-His protein, a 95 amino-acids protein, were selected: Y1775 (Mut⁺ phenotype) and Y1776(Mut^{s} phenotype).

One recombinant strain expressing Nef-Tat mutant-His fusion protein, a 302 amino-acids protein was selected: Y1774(Mut⁺ phenotype).

### Example 3

### Fermentation of Pichia pastoris Producing Recombinant Tat-His

A typical process is described in the table hereafter.

Fermentation includes a growth phase (feeding with a glycerol-based medium according to an appropriate curve) leading to a high cell density culture and an induction phase (feeding with a methanol and a salts/micro-elements solution). During fermentation the growth is followed by taking samples and measuring their absorbance at 620 nm. During the induction phase methanol was added via a pump and its concentration monitored by Gas chromatography (on culture samples) and by on-line gas analysis with a Mass spectrometer. After fermentation the cells were recovered by centrifugation at 5020 g during 30′ at 2-8° C. and the cell paste stored at −20° C. For further work cell paste was thawed, resuspended at an OD (at 620 nm) of 150 in a buffer (Na2HPO4 pH7 50 mM, PMSF 5%, Isopropanol 4 mM) and disrupted by 4 passages in a DynoMill (room 0.6 L, 3000 rpm, 6 L/H, beads diameter of 0.40-0.70 mm).

For evaluation of the expression samples were removed during the induction, disrupted and analyzed by SDS-Page or Western blot. On Coomassie blue stained SDS-gels the recombinant Tat-his was clearly identified as an intense band presenting a maximal intensity after around 72-96H induction.

| | |
|---|---|
| Thawing of a Working seed vial | |
| ↓ | |
| Solid preculture | Synthetic medium: YNB + glucose + agar |
| 30° C., 14-16 H | |
| ↓ | |
| Liquid preculture in two 2 L erlenmeyer | Synthetic medium: 2 × 400 ml YNB + glycerol |
| 30° C., 200 rpm | Stop when OD > 1 (at 620 nm) |
| ↓ | |
| Inoculation of a 20 L fermentor | 5 L initial medium (FSC006AA) |
| | 3 ml antifoam SAG471 (from Witco) |
| | Set-points: Temperature: 30° C. |
| | Overpressure: 0.3 barg |
| | Air flow: 20 Nl/min |
| | Dissolved O2: regulated >40% |
| | pH: regulated at 5 by NH₄OH |
| ↓ | |
| Fed-batch fermentation: growth phase | Feeding with glycerol-based medium FFB005AA |
| Duration around 40 H, | Final OD between 200-500 OD (620 nm) |
| Fed-batch fermentation: induction phase | Feeding with methanol and with a salt/micro-elements |
| Duration: up to 97 H. | solution (FSE021AB). |
| ↓ | |
| Centrifugation | 5020 g/30 min/2-8° C. |
| ↓ | |
| Recover cell paste and store at −20° C. | |
| ↓ | |
| Thaw cells and resuspend at OD150 (620 nm) in buffer | Buffer: Na2HPO4 pH 7 50 mM, PMSF 5%, |
| | Isopropanol 4 mM |
| ↓ | |
| Cell disruption in Dyno-mill | Dyno-mill: (room 0.6 L, 3000 rpm, 6 L/H, beads |
| 4 passages | diameter of 0.40-0.70 mm). |
| ↓ | |
| Transfer for extraction/purification | |

### Media Used for Fermentation:

Solid preculture: (YNB + glucose + agar)

| | | |
|---|---|---|
| Glucose: | 10 | g/l |
| KH2PO4: | 1 | g/l |
| MgSO4•7H2O: | 0.5 | g/l |
| CaCl2•2H2O: | 0.1 | g/l |
| NaCl: | 0.1 | g/l |
| FeCl3•6H2O: | 0.0002 | g/l |
| CuSO4•5H2O: | 0.00004 | g/l |
| ZnSO4•7H2O: | 0.0004 | g/l |
| Na2MoO4•2H2O: | 0.0002 | g/l |
| MnSO4•H2O: | 0.0004 | g/l |
| H3BO3: | 0.0005 | g/l |
| KI: | 0.0001 | g/l |
| CoCl2•6H2O: | 0.00009 | g/l |
| Riboflavine: | 0.000016 | g/l |
| Biotine: | 0.000064 | g/l |
| (NH4)2SO4: | 5 | g/l |
| Acide folique: | 0.000064 | g/l |
| Inositol: | 0.064 | g/l |
| Pyridoxine: | 0.008 | g/l |
| Thiamine: | 0.008 | g/l |
| Niacine: | 0.000032 | g/l |
| Panthoténate Ca: | 0.008 | g/l |
| Para-aminobenzoic acid: | 0.000016 | g/l |
| Agar | 18 | g/l |

Liquid preculture, (YNB + glycerol)

| | | |
|---|---|---|
| Glycerol: | 2% | (v/v) |
| KH2PO4: | 1 | g/l |
| MgSO4•7H2O: | 0.5 | g/l |
| CaCl2•2H2O: | 0.1 | g/l |
| NaCl: | 0.1 | g/l |
| FeCl3•6H2O: | 0.0002 | g/l |
| CuSO4•5H2O: | 0.00004 | g/l |
| ZnSO4•7H2O: | 0.0004 | g/l |
| Na2MoO4•2H2O: | 0.0002 | g/l |
| MnSO4•H2O: | 0.0004 | g/l |
| H3BO3: | 0.0005 | g/l |
| KI: | 0.0001 | g/l |
| CoCl2•6H2O: | 0.00009 | g/l |
| Riboflavine: | 0.000016 | g/l |
| Biotine: | 0.000064 | g/l |
| (NH4)2SO4: | 5 | g/l |
| Acide folique: | 0.000064 | g/l |
| Inositol: | 0.064 | g/l |
| Pyridoxine: | 0.008 | g/l |
| Thiamine: | 0.008 | g/l |
| Niacine: | 0.000032 | g/l |
| Panthoténate Ca: | 0.008 | g/l |
| Para-aminobenzoic acid: | 0.000016 | g/l |

Initial fermentor charge: (FSC006AA)

| | | |
|---|---|---|
| (NH4)₂SO4: | 6.4 | g/l |
| KH2PO4: | 9 | g/l |
| MgSO4•7H2O: | 4.7 | g/l |
| CaCl2•2H2O: | 0.94 | g/l |
| FeCl3•6H2O: | 10 | mg/l |
| HCl: | 1.67 | ml/l |
| CuSO4•5H2O: | 0.408 | mg/l |
| ZnSO4•7H2O: | 4.08 | mg/l |
| Na2MoO4•2H2O: | 2.04 | mg/l |
| MnSO4•H2O: | 4.08 | mg/l |
| H3BO3: | 5.1 | mg/l |
| KI: | 1.022 | mg/l |
| CoCl2•6H2O: | 0.91 | mg/l |
| NaCl: | 0.06 | g/l |
| Biotine: | 0.534 | mg/l |

Feeding solution used for growth phase (FFB005AA)

| | | |
|---|---|---|
| Glycerol: | 38.7% | v/v |
| MgSO4•7H2O: | 13 | g/l |
| CaCl2•2H2O: | 2.6 | g/l |
| FeCl3•6H2O: | 27.8 | mg/l |
| ZnSO4•7H2O | 11.3 | mg/l |
| MnSO4•H2O: | 11.3 | mg/l |
| KH2PO4: | 24.93 | g/l |
| Na2MoO4•2H2O: | 5.7 | mg/l |
| CuSO4•5H2O: | 1.13 | mg/l |
| CoCl2•6H2O: | 2.5 | mg/l |
| H3BO3: | 14.2 | mg/l |
| Biotine: | 1.5 | mg/l |
| KI: | 2.84 | mg/l |
| NaCl: | 0.167 | g/l |

Feeding solution of salts and micro-elements
used during induction (FSE021AB):

| | | |
|---|---|---|
| KH2PO4: | 45 | g/l |
| MgSO4•7H2O: | 23.5 | g/l |
| CaCl2•2H2O: | 4.70 | g/l |
| NaCl: | 0.3 | g/l |
| HCl: | 8.3 | ml/l |
| CuSO4•5H2O: | 2.04 | mg/l |
| ZnSO4•7H2O: | 20.4 | mg/l |
| Na2MoO4•2H2O: | 10.2 | mg/l |
| MnSO4•H2O: | 20.4 | mg/l |
| H3BO3: | 25.5 | mg/l |
| KI: | 5.11 | mg/l |
| CoCl2•6H2O: | 4.55 | mg/l |
| FeCl3•6H2O: | 50.0 | mg/l |
| Biotine: | 2.70 | mg/l |

### Example 4

### Purification of Nef-Tat-His Fusion Protein (Pichia pastoris)

The purification scheme has been developed from 146 g of recombinant Pichia pastoris cells (wet weight) or 2 L Dyno-mill homogenate OD 55. The chromatographic steps are performed at room temperature. Between steps, Nef-Tat positive fractions are kept overnight in the cold room (+4° C.); for longer time, samples are frozen at −20° C.

| | |
|---|---|
| 146 g of Pichia pastoris cells | |
| ⇓ | |
| Homogenization | Buffer: 2 L 50 mM PO₄ pH 7.0 |
| | final OD: 50 |
| ⇓ | |
| Dyno-mill disruption (4 passes) | |
| ⇓ | |
| Centrifugation | JA10 rotor/9500 rpm/30 min/ |
| | room temperature |
| ⇓ | |
| Dyno-mill Pellet | |
| ⇓ | |
| Wash | Buffer: +2 L 10 mM PO₄ pH 7.5 - |
| (1 h - 4° C.) | 150 mM - NaCl 0.5% empigen |
| ⇓ | |
| Centrifugation | JA10 rotor/9500 rpm/30 min/ |
| | room temperature |
| ⇓ | |
| Pellet | |
| ⇓ | |
| Solubilisation | Buffer: +660 ml 10 mM PO₄ pH |
| (O/N - 4° C.) | 7.5 - 150 mM NaCl - 4.0M GuHCl |
| ⇓ | |
| Reduction | +0.2M 2-mercaptoethanesulfonic |
| (4H - room temperature - | acid, sodium salt (powder |
| in the dark) | addition)/pH adjusted to 7.5 |
| | (with 0.5M NaOH solution) before |
| | incubation |
| ⇓ | |
| carbamidomethylation | +0.25M Iodoacetamide (powder |
| (½ h - room temperature - | addition)/pH adjusted to 7.5 |
| in the dark) | (with 0.5 MNaOH solution) before |
| | incubation |
| ⇓ | |
| Immobilized metal ion affinity | Equilibration buffer: 10 mM PO₄ |
| chromatography on Ni⁺⁺-NTA- | pH 7.5 - 150 mM NaCl - 4.0M |
| Agarose | GuHCl |
| (Qiagen - 30 ml of resin) | Washing buffer: |
| | 1) Equilibration |
| | buffer |
| | 2) 10 mM PO₄ |
| | pH 7.5 - 150 mM NaCl - 6M Urea |
| | 3) 10 mM PO₄ |
| | pH 7.5 - 150 mM NaCl - 6M Urea - |
| | 25 mM Imidazol |
| | Elution buffer: 10 mM PO₄ pH 7.5 - |
| | 150 mM NaCl - 6M Urea -0.5M |
| | Imidazol |
| ⇓ | |
| Dilution | Down to an ionic strength of 18 mS/cm² |
| | Dilution buffer: 10 mM PO₄ pH |
| | 7.5 - 6M Urea |
| ⇓ | |
| Cation exchange | Equilibration buffer: 10 mM PO₄ |
| chromatography | pH 7.5 - 150 mM NaCl - 6.0M |
| on SP Sepharose FF | Urea |
| (Pharmacia - 30 ml of resin) | Washing buffer: |
| | 1) Equilibration |
| | buffer |
| | 2) 10 mM PO₄ |
| | pH 7.5 - 250 mM NaCl - 6M Urea |
| | Elution buffer: 10 mM Borate pH |
| | 9.0 - 2M NaCl - 6M Urea |
| ⇓ | |
| Concentration | up to 5 mg/ml |
| | 10 kDa Omega membrane(Filtron) |
| ⇓ | |
| Gel filtration chromatography on | Elution buffer: 10 mM PO₄ pH 7.5 - |
| Superdex200 XK 16/60 | 150 mM NaCl - 6M Urea |
| (Pharmacia - 120 ml of resin) | 5 ml of sample/injection [symbol] 5 |
| | injections |
| ⇓ | |
| Dialysis | Buffer: 10 mM PO₄ pH 6.8 - |
| (O/N - 4° C.) | 150 mM NaCl - 0.5M Arginin* |
| ⇓ | |
| Sterile filtration | Millex GV 0.22 μm |

*ratio: 0.5M Arginin for a protein concentration of 1600 μg/ml.

### Purity

The level of purity as estimated by SDS-PAGE is shown in FIG. 3 by Daiichi Silver Staining and in FIG. 4 by Coomassie blue G250.
After Superdex200 step: >95%
After dialysis and sterile filtration steps: >95%

### Recovery

51 mg of Nef-Tat-his protein are purified from 146 g of recombinant Pichia pastoris cells (=2 L of Dyno-mill homogenate OD 55)

### Example 5

### Purification of Oxidized Nef-Tat-His Fusion Protein in Pichia pastoris

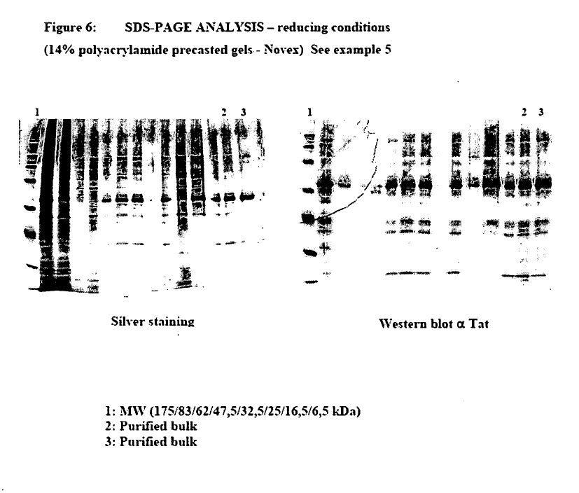

The purification scheme has been developed from 73 g of recombinant Pichia pastoris cells (wet weight) or 1 L Dyno-mill homogenate OD 50. The chromatographic steps are performed at room temperature. Between steps, Nef-Tat positive fractions are kept overnight in the cold room (+4° C.); for longer time, samples are frozen at −20° C.

| | |
|---|---|
| 73 g of Pichia pastoris cells | |
| ⇓ | |
| Homogenization | Buffer: 1 L 50 mM PO₄ pH 7.0 - |
| Pefabloc 5 mM | |
| | final OD: 50 |
| ⇓ | |
| Dyno-mill disruption (4 passes) | |
| ⇓ | |
| Centrifugation | JA10 rotor/9500 rpm/30 min/room |
| | temperature |
| ⇓ | |
| Dyno-mill Pellet | |
| ⇓ | |
| Wash | Buffer: +1 L 10 mM PO₄ pH 7.5 - 150 mM |
| (2 h - 4° C.) | NaCl - 0.5% Empigen |
| ⇓ | |
| Centrifugation | JA10 rotor/9500 rpm/30 min/room |
| | temperature |
| ⇓ | |
| Pellet | |
| ⇓ | |
| Solubilisation | Buffer: +330 ml 10 mM PO₄ pH 7.5 - |
| (O/N - 4° C.) | 150 mM NaCl - 4.0M GuHCl |
| ⇓ | |
| Immobilized metal ion affinity | Equilibration buffer: 10 mM PO₄ pH 7.5 - |
| chromatography on Ni⁺⁺-NTA-Agarose | 150 mM NaCl - 4.0 M GuHCl |
| (Qiagen - 15 ml of resin) | Washing buffer: 1) Equilibration buffer |
| | 2) 10 mM PO₄ pH 7.5 - |
| | 150 mM NaCl - 6 M |
| | Urea |
| | 3) 10 mM PO₄ pH 7.5 - |
| | 150 mM NaCl - 6 M |
| | Urea - 25 mM Imidazol |
| | Elution buffer: 10 mM PO₄ pH 7.5 - |
| | 150 mM NaCl - 6 M Urea - 0.5 M |
| | Imidazol |
| ⇓ | |
| Dilution | Down to an ionic strength of 18 mS/cm² |
| | Dilution buffer: 10 mM PO₄ pH 7.5 - 6 M |
| | Urea |
| ⇓ | |
| Cation exchange chromatography on SP | Equilibration buffer: 10 mM PO₄ pH |
| Sepharose FF | 7.5 - 150 mM NaCl - 6.0 M Urea |
| (Pharmacia - 7 ml of resin) | Washing buffer: 1) Equilibration buffer |
| | 2) 10 mM PO₄ pH 7.5 - |
| | 250 mM NaCl - 6 M |
| | Urea |
| | Elution buffer: 10 mM Borate pH 9.0 - |
| | 2 M NaCl - 6 M Urea |
| ⇓ | |
| Concentration | up to 0.8 mg/ml |
| | 10 kDa Omega membrane(Filtron) |
| ⇓ | |
| Dialysis | Buffer: 10 mM PO₄ pH 6.8 - 150 mM |
| (O/N - 4° C.) | NaCl - 0.5 M Arginin |
| ⇓ | |
| Sterile filtration | Millex GV 0.22 μm |

[symbol] Level of Purity Estimated by SDS-PAGE is Shown in FIG. 6 (Daiichi Silver Staining, Coomassie Blue G250, Western Blotting):
After dialysis and sterile filtration steps: >95%

[symbol] Recovery (Evaluated by a Calorimetric Protein Assay: DOC TCA BCA)
2,8 mg of oxidized Nef-Tat-his protein are purified from 73 g of recombinant Pichia pastoris cells (wet weight) or 1 L of Dyno-mill homogenate OD 50.

### Example 6

### Purification of Reduced Tat-His Protein (Pichia pastoris)

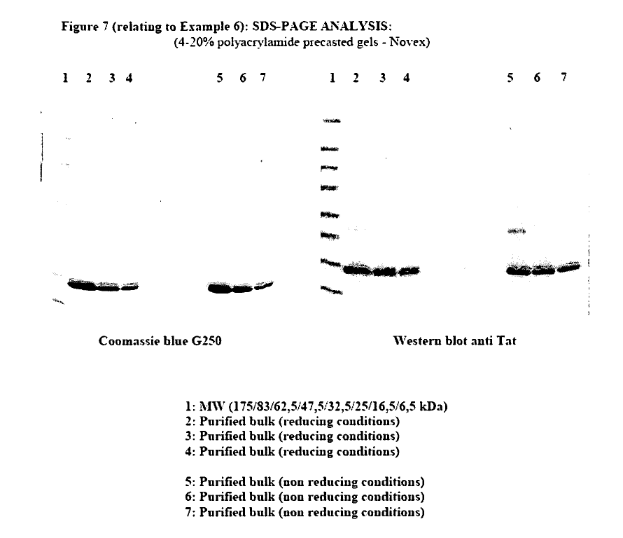

The purification scheme has been developed from 160 g of recombinant Pichia pastoris cells (wet weight) or 2 L Dyno-mill homogenate OD 66. The chromatographic steps are performed at room temperature. Between steps, Tat positive fractions are kept overnight in the cold room (+4° C.); for longer time, samples are frozen at −20° C.

| | |
|---|---|
| 160 g of Pichia pastoris cells | |
| ⇓ | |
| Homogenization | Buffer: +2 L 50 mM PO₄ pH 7.0 - 4 mM PMSF |
| | final OD: 66 |
| ⇓ | |
| Dyno-mill disruption (4 passes) | |
| ⇓ | |
| Centrifugation | JA10 rotor/9500 rpm/30 min/room temperature |
| ⇓ | |
| Dyno-mill Pellet | |
| ⇓ | |
| Wash | Buffer: +2 L 10 mM PO₄ pH 7.5 - 150 mM NaCl - |
| (1 h - 4° C.) | 1% Empigen |
| ⇓ | |
| Centrifugation | JA10 rotor/9500 rpm/30 min/room temperature |
| ⇓ | |
| Pellet | |
| ⇓ | |
| Solubilisation | Buffer: +660 ml 10 mM PO₄ pH 7.5 - 150 mM |
| (O/N - 4° C.) | NaCl - 4.0 M GuHCl |
| ⇓ | |
| Centrifugation | JA10 rotor/9500 rpm/30 min/room temperature |
| ⇓ | |
| Reduction | +0.2 M 2-mercaptoethanesulfonic acid, sodium |
| (4 H - room temperature - in the dark) | salt (powder addition)/pH adjusted to 7.5 (with |
| | 1 M NaOH solution) before incubation |
| ⇓ | |
| carbamidomethylation | +0.25 M Iodoacetamide (powder addition)/pH |
| (½ h - room temperature - in the dark) | adjusted to 7.5 (with 1 M NaOH solution) before |
| | incubation |
| ⇓ | |
| Immobilized metal ion affinity | Equilibration buffer: 10 mM PO₄ pH 7.5 - 150 mM |
| chromatography on Ni⁺⁺-NTA-Agarose | NaCl - 4.0 M GuHCl |
| (Qiagen - 60 ml of resin) | Washing buffer: 1) Equilibration buffer |
| | 2) 10 mM PO₄ pH 7.5 - 150 mM |
| | NaCl - 6 M Urea |
| | 3) 10 mM PO₄ pH 7.5 - 150 mM |
| | NaCl - 6 M Urea - 35 mM |
| | Imidazol |
| | Elution buffer: 10 mM PO₄ pH 7.5 - 150 mM NaCl - |
| | 6 M Urea - 0.5 M Imidazol |
| ⇓ | |
| Dilution | Down to an ionic strength of 12 mS/cm |
| | Dilution buffer: 20 mM Borate pH 8.5 - 6 M Urea |
| ⇓ | |
| Cation exchange chromatography on SP | Equilibration buffer: 20 mM Borate pH 8.5 - |
| Sepharose FF | 150 mM NaCl - 6.0 M Urea |
| (Pharmacia - 30 ml of resin) | Washing buffer: Equilibration buffer |
| | Elution buffer: 20 mM Borate pH 8.5 - 400 mM |
| | NaCl - 6.0 M Urea |
| ⇓ | |
| Concentration | up to 1.5 mg/ml |
| | 10 kDa Omega membrane(Filtron) |
| ⇓ | |
| Dialysis | Buffer: 10 mM PO₄ pH 6.8 - 150 mM NaCl - |
| (O/N - 4° C.) | 0.5 M Arginin |
| ⇓ | |
| Sterile filtration | Millex GV 0.22 μm |

[symbol] Level of Purity Estimated by SDS-PAGE as Shown in FIG. 7(Daiichi Silver Staining, Coomassie Blue G250, Western Blotting):
After dialysis and sterile filtration steps: >95%

[symbol] Recovery (Evaluated by a Calorimetric Protein Assay: DOC TCA BCA)

48 mg of reduced Tat-his protein are purified from 160 g of recombinant Pichia pastoris cells (wet weight) or 2 L of Dyno-mill homogenate OD 66.

### Example 7

### Purification of Oxidized Tat-His Protein (Pichia pastoris)

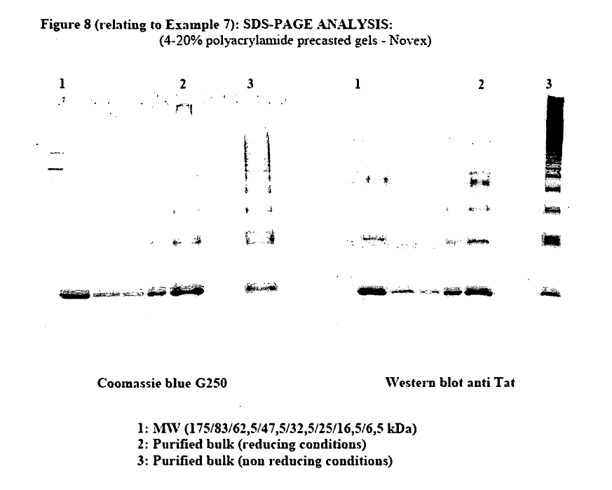

The purification scheme has been developed from 74 g of recombinant Pichia pastoris cells (wet weight) or IL Dyno-mill homogenate OD60. The chromatographic steps are performed at room temperature. Between steps, Tat positive fractions are kept overnight in the cold room (+4° C.); for longer time, samples are frozen at −20° C.

| | |
|---|---|
| 74 g of Pichia pastoris cells | |
| ⇓ | |
| Homogenization | Buffer: +1 L 50 mM PO₄ pH 7.0 - 5 mM Pefabloc |
| | final OD: 60 |
| ⇓ | |
| Dyno-mill disruption (4 passes) | |
| ⇓ | |
| Centrifugation | JA10 rotor/9500 rpm/30 min/room temperature |
| ⇓ | |
| Dyno-mill Pellet | |
| ⇓ | |
| Wash | Buffer: +1 L 10 mM PO₄ pH 7.5 - 150 mM NaCl - |
| (1 h - 4° C.) | 1% Empigen |
| ⇓ | |
| Centrifugation | JA10 rotor/9500 rpm/30 min/room temperature |
| ⇓ | |
| Pellet | |
| ⇓ | |
| Solubilisation | Buffer: +330 ml 10 mM PO₄ pH 7.5 - 150 mM |
| (O/N - 4° C.) | NaCl - 4.0 M GuHCl |
| ⇓ | |
| Centrifugation | JA10 rotor/9500 rpm/30 min/room temperature |
| ⇓ | |
| Immobilized metal ion affinity | Equilibration buffer: 10 mM PO₄ pH 7.5 - 150 mM |
| chromatography on Ni⁺⁺-NTA-Agarose | NaCl - 4.0 M GuHCl |
| (Qiagen - 30 ml of resin) | Washing buffer: 1) Equilibration buffer |
| | 2) 10 mM PO₄ pH 7.5 - 150 mM |
| | NaCl - 6 M Urea |
| | 3) 10 mM PO₄ pH 7.5 - 150 mM |
| | NaCl - 6 M Urea - 35 mM |
| | Imidazol |
| | Elution buffer: 10 mM PO₄ pH 7.5 - 150 mM |
| | NaCl - 6 M Urea - 0.5 M Imidazol |
| ⇓ | |
| Dilution | Down to an ionic strength of 12 mS/cm |
| | Dilution buffer: 20 mM Borate pH 8.5 - 6 M Urea |
| ⇓ | |
| Cation exchange chromatography on SP | Equilibration buffer: 20 mM Borate pH 8.5 - |
| Sepharose FF | 150 mM NaCl - 6.0 M Urea |
| (Pharmacia - 15 ml of resin) | Washing buffer: 1) Equilibration buffer |
| | 2) 20 mM Borate pH 8.5 - |
| | 400 mM NaCl - 6.0 M Urea |
| | Elution buffer: 20 mM Piperazine pH 11.0 - 2 M |
| | NaCl - 6 M Urea |
| ⇓ | |
| Concentration | up to 1.5 mg/ml |
| | 10 kDa Omega membrane(Filtron) |
| ⇓ | |
| Dialysis | Buffer: 10 mM PO₄ pH 6.8 - 150 mM NaCl - |
| (O/N - 4° C.) | 0.5 M Arginin |
| ⇓ | |
| Sterile filtration | Millex GV 0.22 μm |

[symbol] Level of Purity Estimated by SDS-PAGE as Shown in FIG. 8 (Daiichi Silver Coomassie Blue G250 Western Blotting):
After dialysis and sterile filtration steps: >95%

[symbol] Recovery (Evaluated by a Calorimetric Protein Assay: DOC TCA BCA)

19 mg of oxidized Tat-his protein are purified from 74 g of recombinant Pichia pastoris cells (wet weight) or 1 L of Dyno-mill homogenate OD 60.

### Example 8

### Purification of SIV Reduced Nef-His Protein (Pichia pastoris)

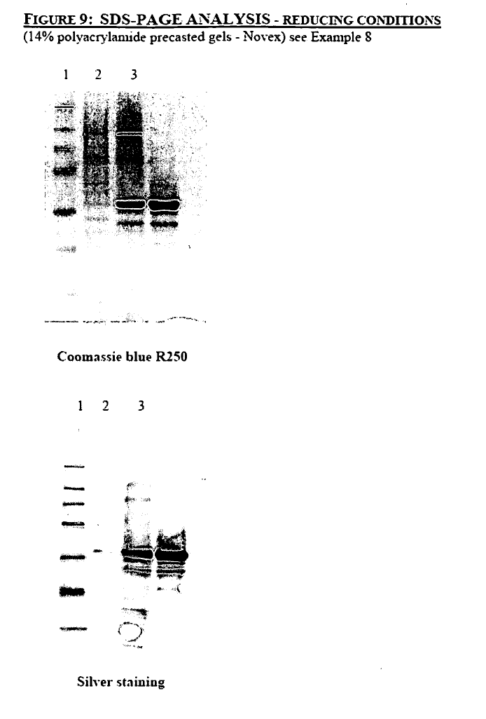

The purification scheme has been developed from 340 g of recombinant Pichia pastoris cells (wet weight) or 4 L Dyno-mill homogenate OD 100. The chromatographic steps are performed at room temperature. Between steps, Nef positive fractions are kept overnight in the cold room (+4° C.); for longer time, samples are frozen at −20° C.

| | |
|---|---|
| 340 g of Pichia pastoris cells | |
| ⇓ | |
| Homogenization | Buffer: 4 L 50 mM PO₄ pH 7.0 - PMSF 4 mM |
| | final OD: 100 |
| ⇓ | |
| Dyno-mill disruption (4 passes) | |
| ⇓ | |
| Centrifugation | JA10 rotor/9500 rpm/60 min/room |
| | temperature |
| ⇓ | |
| Dyno-mill Pellet | |
| ⇓ | |
| Solubilisation | Buffer: +2.6 L 10 mM PO₄ pH 7.5 - 150 mM |
| (O/N - 4° C.) | NaCl - 4.0 M GuHCl |
| ⇓ | |
| Centrifugation | JA10 rotor/9500 rpm/30 min/room |
| | temperature |
| ⇓ | |
| Reduction | +0.2 M 2-mercaptoethanesulfonic acid, sodium |
| (4 H - room temperature - in the dark) | salt (powder addition)/pH adjusted to 7.5 (with |
| | 1 M NaOH solution) before incubation |
| ⇓ | |
| Carbamidomethylation | +0.25 M Iodoacetamide (powder addition)/pH |
| (½ h - room temperature - in the dark) | adjusted to 7.5 (with 1 M NaOH solution) |
| | before incubation |
| ⇓ | |
| Immobilized metal ion affinity | Equilibration buffer: 10 mM PO₄ pH 7.5 - 150 mM |
| chromatography on Ni⁺⁺-NTA-Agarose | NaCl - 4.0 M GuHCl |
| (Qiagen - 40 ml of resin) | Washing buffer: 1) Equilibration buffer |
| | 2) 10 mM PO₄ pH 7.5 - |
| | 150 mM NaCl - 6 M Urea - |
| | 25 mM Imidazol |
| | Elution buffer: 10 mM PO₄ pH 7.5 - 150 mM |
| | NaCl - 6 M Urea - 0.5 M Imidazol |
| ⇓ | |
| Concentration | up to 3 mg/ml |
| | 10 kDa Omega membrane(Filtron) |
| ⇓ | |
| Gel filtration chromatography on | Elution buffer: 10 mM PO₄ pH 7.5 - 150 mM |
| Superdex 200 | NaCl - 6 M Urea |
| (Pharmacia - 120 ml of resin) | |
| ⇓ | |
| Concentration | up to 1.5 mg/ml |
| | 10 kDa Omega membrane(Filtron) |
| ⇓ | |
| Dialysis | Buffer: 10 mM PO₄ pH 6.8 - 150 mM NaCl - |
| (O/N - 4° C.) | Empigen 0.3% |
| ⇓ | |
| Sterile filtration | Millex GV 0.22 μm |

[symbol] Level of Purity Estimated by SDS-PAGE as Shown in FIG. 9 (Daiichi Silver Staining, Coomassie Blue G250, Western Blotting):
After dialysis and sterile filtration steps: >95%

[symbol] Recovery (Evaluated by a Calorimetric Protein Assay: DOC TCA BCA)
20 mg of SIV reduced Nef-his protein are purified from 340 g of recombinant Pichia pastoris cells (wet weight) or 4 L of Dyno-mill homogenate OD 100.

### Example 9

### Purification of HIV Reduced Nef-His Protein (Pichia pastoris)

The purification scheme has been developed from 160 g of recombinant Pichia pastoris cells (wet weight) or 3 L Dyno-mill homogenate OD 50. The chromatographic steps are performed at room temperature. Between steps, Nef positive fractions are kept overnight in the cold room (+4° C.); for longer time, samples are frozen at −20° C.

| | |
|---|---|
| 160 g of Pichia pastoris cells | |
| ⇓ | |
| Homogenization | Buffer: 3 L 50 mM PO₄ pH 7.0 - Pefabloc 5 mM |
| | final OD: 50 |
| ⇓ | |
| Dyno-mill disruption (4 passes) | |
| ⇓ | |
| Freezing/Thawing | |
| ⇓ | |
| Centrifugation | JA10 rotor/9500 rpm/60 min/room |
| | temperature |
| ⇓ | |
| Dyno-mill Pellet | |
| ⇓ | |
| Solubilisation | Buffer: +1 L 10 mM PO₄ pH 7.5 - 150 mM |
| (O/N - 4° C.) | NaCl - 4.0 M GuHCl |
| ⇓ | |
| Centrifugation | JA10 rotor/9500 rpm/60 min/room |
| | temperature |
| ⇓ | |
| Reduction | +0.1 M 2-mercaptoethanesulfonic acid, sodium |
| (3 H - room temperature - in the dark) | salt (powder addition)/pH adjusted to 7.5 (with |
| | 1 M NaOH solution) before incubation |
| ⇓ | |
| Carbamidomethylation | +0.15 M Iodoacetamide (powder addition)/pH |
| (½ h - room temperature - in the dark) | adjusted to 7.5 (with 1 M NaOH solution) |
| | before incubation |
| ⇓ | |
| Immobilized metal ion affinity | Equilibration buffer: 10 mM PO₄ pH 7.5 - 150 mM |
| chromatography on Ni⁺⁺-NTA-Agarose | NaCl - 4.0 M GuHCl |
| (Qiagen - 10 ml of resin) | Washing buffer: 1) Equilibration buffer |
| | 2) 10 mM PO₄ pH 7.5 - |
| | 150 mM NaCl - 6 M Urea |
| | 3) 10 mM PO₄ pH 7.5 - |
| | 150 mM NaCl - 6 M Urea - |
| | 25 mM Imidazol |
| | Elution buffer: 10 mM Citrate pH 6.0 - 150 mM |
| | NaCl - 6 M Urea - 0.5 M Imidazol |
| ⇓ | |
| Concentration | up to 3 mg/ml |
| | 10 kDa Omega membrane(Filtron) |
| ⇓ | |
| Gel filtration chromatography on | Elution buffer: 10 mM PO₄ pH 7.5 - 150 mM |
| Superdex 200 | NaCl - 6 M Urea |
| (Pharmacia - 120 ml of resin) | |
| ⇓ | |
| Dialysis | Buffer: 10 mM PO₄ pH 6.8 - 150 mM NaCl - |
| (O/N - 4° C.) | 0.5 M Arginin |
| ⇓ | |
| Sterile filtration | Millex GV 0.22 μm |

[symbol] Level of Purity Estimated by SDS-PAGE as Shown in FIG. 10 (Daiichi Silver Staining, Coomassie Blue G250, Western Blotting):
After dialysis and sterile filtration steps: >95%

[symbol] Recovery (Evaluated by a Calorimetric Protein Assay: DOC TCA BCA)
20 mg of HIV reduced Nef-his protein are purified from 160 g of recombinant Pichia pastoris cells (wet weight) or 3 L of Dyno-mill homogenate OD 50.

### Example 10

### Expression of SIV Nef Sequence in Pichia pastoris

In order to evaluate Nef and Tat antigens in the pathogenic SHIV challenge model, we have expressed the Nef protein of simian immunodeficiency virus (SIV) of macaques, SIVmac239 (Aids Research and Human Retroviruses, 6:1221-1231, 1990). In the Nef coding region, SIV mac 239 has an in-frame stop codon after 92aa predicting a truncated product of only 10 kD. The remainder of the Nef reading frame is open and would be predicted to encode a protein of 263aa (30 kD) in its fully open form.

Our starting material for SIVmac239 nefgene was a DNA fragment corresponding to the complete coding sequence, cloned on the LX5N plasmid (received from Dr R. C. Desrosiers, Southborough, Mass., USA).

This SIV nef gene is mutated at the premature stop codon (nucleotide G at position 9353 replaces the original T nucleotide) in order to express the full-length SIVmac239 Nef protein.

To express this SIV nef gene in Pichia pastoris, the PHIL-D2-MOD Vector (previously used for the expression of HIV-1 nef and tat sequences) was used. The recombinant protein is expressed under the control of the inducible alcohol oxidase (AOX1) promoter and the c-terminus of the protein is elongated by a Histidine affinity tail that will facilitate the purification.

10.1 Construction of the Integrative Vector pRIT 14908

To construct pRIT 14908, the SIV nef gene was amplified by PCR from the pLX5N/SIV-NEF plasmid with primers SNEF1 and SNEF2.

```
PRIMER SNEF1: 5′ATCGTCCATG.GGTGGAGCTATTTT 3′
                                          NcoI
PRIMER SNEF2: 5′CGGCTACTAGTGCGAGTTTCCTT 3′
                                          SpeI
```

The SIV nef DNA region amplified starts at nucleotide 9077 and terminates at nucleotide 9865 (Aids Research and Human Retroviruses, 6:1221-1231, 1990).

An NcoI restriction site (with carries the ATG codon of the nef gene) was introduced at the 5′ end of the PCR fragment while a SpeI site was introduced at the 3′ end. The PCR fragment obtained and the integrative PHIL-D2-MOD vector were both restricted by NcoI and SpeI. Since one NcoI restriction site is present on the SIV nef amplified sequence (at position 9286), two fragments of respectively ±200 bp and ±600 bp were obtained, purified on agarose gel and ligated to PHIL-D2-MOD vector. The resulting recombinant plasmid received, after verification of the nef amplified region by automated sequencing, the pRIT 14908 denomination.

10.2 Transformation of Pichia pastoris Strain GS115(his4).

To obtain Pichia pastoris strain expressing SIV nef-His, strain GS115 was transformed with a linear NotI fragment carrying only the expression cassette and the HIS4 gene (FIG. 11).

This linear NotI DNA fragment with homologies at both ends with AOX1 resident P. pastoris gene, favors recombination at the AOX1 locus.

Multicopy integrant clones were selected by quantitative dot blot analysis.

One transformant showing the best production level for the recombinant protein was selected and received the Y1772 denomination.

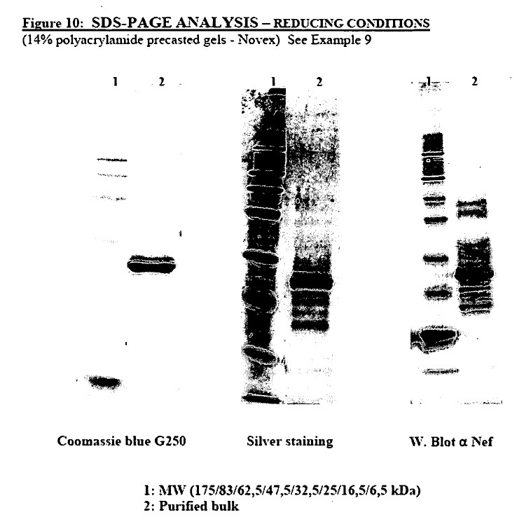

Strain Y1772 produces the recombinant SIV Nef-His protein, a 272 amino acids protein which would be composed of:
Myristic acid
A methionine, created by the use of NcoI cloning site of PHIL-D2-MOD vector.
262 amino acids (aa) of Nef protein (starting at aa 2 and extending to aa 263, see FIG. 12)
A threonine and a serine created by the cloning procedure (cloning at SpeI site of PHIL-D2-MOD vector (FIG. 11).
One glycine and six histidines.

Nucleic and Protein sequences are shown on FIG. 12.

### 10.3 Characterization of the Expressed Product of Strain Y1772.

### Expression Level

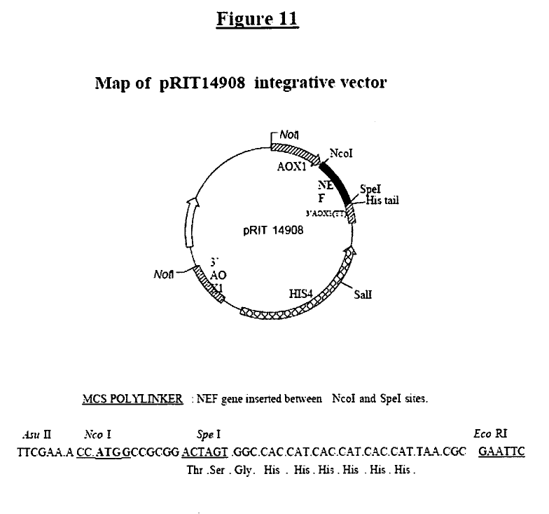

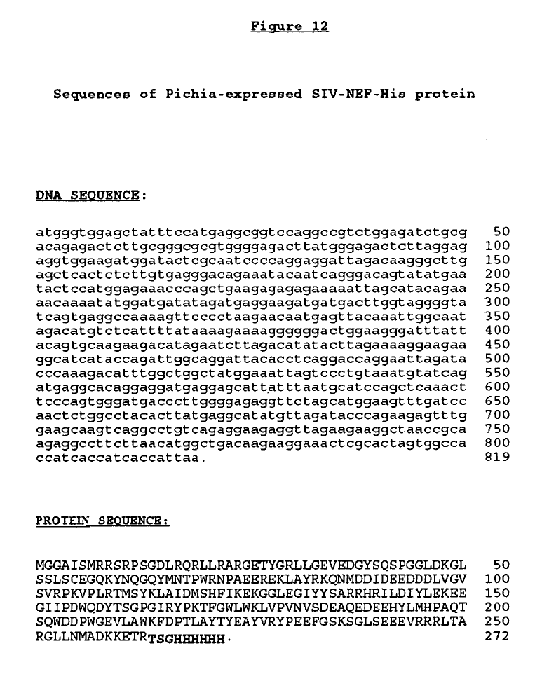

After 16 hours induction in medium containing 1% methanol as carbon source, abundance of the recombinant Nef-His protein, was estimated at 10% of total protein (FIG. 13, lanes 3-4).

### Solubility

Induced cultures of recombinant strain Y1772 producing the Nef-His protein were centrifuged. Cell pellets were resuspended in breaking buffer, disrupted with 0.5 mm glass beads and the cell extracts were centrifuged. The proteins contained in the insoluble pellet (P) and in the soluble supernatant (S) were compared on a Coomassie Blue stained SDS-PAGE10%.

As shown in FIG. 13, the majority of the recombinant protein from strain Y1772 (lanes 3-4) is associated with the insoluble fraction.

Strain Y1772 which presents a satisfactory recombinant protein expression level is used for the production and purification of SIV Nef-His protein.

### Example 11

### Expression of GP120 in CHO

A stable CHO-K1 cell line which produces a recombinant gP120 glycoprotein has been established. Recombinant gP120 glycoprotein is a recombinant truncated form of the gP120 envelope protein of HIV-1 isolate W61D. The protein is excreted into the cell culture medium, from which it is subsequently purified.

Construction of gp120 Transfection Plasmid pRIT13968

The envelope DNA coding sequence (including the 5′ exon of tat and rev) of HIV-1 isolate W61D was obtained (Dr. Tersmette, CCB, Amsterdam) as a genomic gp160 envelope containing plasmid W61D (Nco-XhoI). The plasmid was designated pRIT13965.

In order to construct a gp120 expression cassette a stop codon had to be inserted at the amino acid glu515 codon of the gp160 encoding sequence in pRIT13965 using a primer oligonucleotide sequence (DIR 131) and PCR technology. Primer DIR 131 contains three stop codons (in all open reading frames) and a SalI restriction site.

The complete gp120 envelope sequence was then reconstituted from the N-terminal BamH1-DraI fragment (170 bp) of a gp160 plasmid subclone pW61d env (PRIT13966) derived from pRIT13965, and the DraI-SalI fragment (510 bp) generated by PCR from pRIT13965. Both fragments were gel purified and ligated together into the E. coli plasmid pUC18, cut first by SalI (klenow treated), and then by BamH1. This resulted in plasmid pRIT13967. The gene sequence of the XmaI-SalI fragment (1580 bp) containing the gp120 coding cassette was sequenced and found to be identical to the predicted sequence. Plasmid RIT13967 was ligated into the CHO GS-expression vector pEE14 (Celltech Ltd., UK) by cutting first with BclI (klenow treated) and then by XmaI. The resulting plasmid was designated pRIT13968.

### Preparation of Master Cell Bank

The gp120-construct (pRIT13968) was transfected into CHO cells by the classical CaPO₄-precipitation/glycerol shock procedure. Two days later the CHOK1 cells were subjected to selective growth medium (GMEM+methionine sulfoximine (MSX) 25 μM+Glutamate+asparagine+10% Foetal calf serum). Three chosen transfectant clones were further amplified in 175 m² flasks and few cell vials were stored at −80° C. C-env 23,9 was selected for further expansion.

A small prebank of cells was prepared and 20 ampoules were frozen. For preparation of the prebank and the MCB, cells were grown in GMEM culture medium, supplemented with 7.5% fetal calf serum and containing 50 μM MSX. These cell cultures were tested for sterility and mycoplasma and proved to be negative.

The Master Cell Bank CHOKI env 23.9 (at passage 12) was prepared using cells derived from the premaster cell bank. Briefly, two ampoules of the premaster seed were seeded in medium supplemented with 7.5% dialysed foetal bovine serum. The cells were distributed in four culture flasks and cultured at 37° C. After cell attachment the culture medium was changed with fresh medium supplemented with 50 μM MSX. At confluence, cells were collected by trypsination and subcultured with a ⅛ split ratio in T-flasks—roller bottle—cell factory units. Cells were collected from cell factory units by trypsination and centrifugation. The cell pellet was resuspended in culture medium supplemented with DMSO as cryogenic preservative. Ampoules were prelabelled, autoclaved and heat-sealed (250 vials). They were checked for leaks and stored overnight at −70° C. before storage in liquid nitrogen.

### Cell Culture and Production of Crude Harvest

Two vials from a master cell bank are thawed rapidly. Cells are pooled and inoculated in two T-flasks at 37°±1° C. with an appropriate culture medium supplemented with 7.5% dialysed foetal bovine (FBS) serum. When reaching confluence (passage 13), cells are collected by trypsinisation, pooled and expanded in 10 T-flasks as above. Confluent cells (passage 14) are trypsinised and expanded serially in 2 cell factory units (each 6000 cm²; passage 15), then in 10 cell factories (passage 16). The growth culture medium is supplemented with 7.5% dialysed foetal bovine (FBS) serum and 1% MSX. When cells reach confluence, the growth culture medium is discarded and replaced by “production medium” containing only 1% dialysed foetal bovine serum and no MSX. Supernatant is collected every two days (48 hrs-interval) for up to 32 days. The harvested culture fluids are clarified immediately through a 1.2-0.22 μm filter unit and kept at −20° C. before purification.

### Example 12

### Purification of HIV gP120 (W61D CHO) from Cell Culture Fluid

All purification steps are performed in a cold room at 2-8° C. pH of buffers are adjusted at this temperature and are filtered on 0.2 μm filter. They are tested for pyrogen content (LAL assay). Optical density at 280 nm, pH and conductivity of column eluates are continuously monitored.

### (i) Clarified Culture Fluid

The harvested clarified cell culture fluid (CCF) is filter-sterilized and Tris buffer, pH 8.0 is added to 30 mM final concentration. CCF is stored frozen at −20° C. until purification.

### (ii) Hydrophobic Interaction Chromatography

After thawing, ammonium sulphate is added to the clarified culture fluid up to 1 M. The solution is passed overnight on a TSK/TOYOPEARL-BUTYL 650 M (TOSOHAAS) column, equilibrated in 30 mM Tris buffer-pH 8.0-1 M ammonium sulphate. Under these conditions, the antigen binds to the gel matrix. The column is washed with a decreasing stepwise ammonium sulphate gradient. The antigen is eluted at 30 mM Tris buffer-pH 8.0-0.25 M ammonium sulphate.

(iii) Anion-Exchange Chromatography

After reducing the conductivity of the solution between 5 and 6 mS/cm, the gP120 pool of fractions is loaded onto a Q-sepharose Fast Flow (Pharmacia) column, equilibrated in Tris-saline buffer—pH 8.0. The column is operated on a negative mode, i.e. gP120 does not bind to the gel, while most of the impurities are retained.

### (iv) Concentration and Diafiltration by Ultrafiltration

In order to increase the protein concentration, the gP120 pool is loaded on a FILTRON membrane “Omega Screen Channel”, with a 50 kDa cut-off. At the end of the concentration, the buffer is exchanged by diafiltration with 5 mM phosphate buffer containing CaCl₂ 0.3 mM, pH 7.0. If further processing is not performed immediately, the gP120 pool is stored frozen at −20° C. After thawing the solution is filtered onto a 0.2 μM membrane in order to remove insoluble materiel.

### (v) Chromatography on Hydroxyapatite

The gP120 UF pool is loaded onto a macro-Prep Ceramic Hydroxyapatite, type II (Biorad) column equilibrated in 5 mM phosphate buffer+CaCl₂ 0.3 mM, pH 7.0. The column is washed with the same buffer. The antigen passes through the column and impurities bind to the column.

### (vi) Cation Exchange Chromatography

The gP120 pool is loaded on a CM/TOYOPEARL-650 S (TOSOHAAS) column equilibrated in acetate buffer 20 mM, pH 5.0. The column is washed with the same buffer, then acetate 20 mM, pH 5.0 and NaCl 10 mM. The antigen is then eluted by the same buffer containing 80 mM NaCl.

(vii) Ultrafiltration

In order to augment the virus clearance capacity of the purification process, an additional ultrafiltration step is carried out. The gP120 pool is subjected to ultrafiltration onto a FILTRON membrane “Omega Screen Channel”, cut-off 150 kDa. This pore-size membrane does not retain the antigen. After the process, the diluted antigen is concentrated on the same type of membrane (Filtron) but with a cut-off of 50 kDa.

(viii) Size Exclusion Gel Chromatography

The gP120 pool is applied to a SUPERDEX 200 (PHARMACIA) column in order to exchange the buffer and to eliminate residual contaminants. The column is eluted with phosphate buffer saline (PBS).

### (ix) Sterile Filtration and Storage

Fractions are sterilized by filtration on a 0.2 μM PVDF membrane (Millipore). After sterile filtration, the purified bulk is stored frozen at −20° C. up to formulation. The purification scheme is summarized by the flow sheet below.

[symbol] Level of purity of the purified bulk estimated by SDS-PAGE analysis (Silver staining/Coomassie Blue/Western Blotting) is ≧95%.

[symbol] Production yeild is around 2.5 mg/L CCF (according to Lowery assay)—Global purification yeild is around 25% (according to Elisa assay)

[symbol] Purified material is stable 1 week at 37° C. (according to WB analysis)

Purification of gp120 from culture fluid
Mark ✓ indicate steps that are critical for virus removal.

| | |
|---|---|
| CLARIFIED CULTURE FLUID | |
| ↓ | |
| HYDROPHOBIC INTERACTION CHROMATOGRAPHY | |
| (BUTYL - TOYOPEARL 650 - ) | |
| ↓ | |
| ANION EXCHANGE CHROMATOGRAPHY | ✓ |
| (NEGATIVE MODE) | |
| (Q-SEPHAROSE) | |
| ↓ | |
| 50 KD ULTRAFILTRATION | |
| (CONCENTRATION AND BUFFER EXCHANGE) | |
| ↓ | |
| (STORAGE −20° C.) | |
| ↓ | |
| HYDROXYAPATITE CHROMATOGRAPHY | |
| (NEGATIVE MODE) | |
| (MACROPREP CERAMIC HYDROXYAPATITE II) | |
| ↓ | |
| CATION EXCHANGE CHROMATOGRAPHY | |
| (CM-TOYOPEARL 650 S) | |
| ↓ | |
| 150 KD ULTRAFILTRATION | ✓ |
| (OMEGA MEMBRANES/FILTRON) | |
| ↓ | |
| 50 KD ULTRAFILTRATION | |
| (CONCENTRATION) | |
| ↓ | |
| SIZE EXCLUSION CHROMATOGRAPHY | ✓ |
| (SUPERDEX 200) | |
| STERILE FILTRATION | |
| ↓ | |
| PURIFIED BULK | |
| STORAGE −20° C. | |

### Example 13

### Vaccine Preparation

A vaccine prepared in accordance with the invention comprises the expression products of one or more DNA recombinants encoding an antigen. Furthermore, the formulations comprise a mixture of 3 de-O-acylated monophosphoryl lipid A 3D-MPL and QS21 in an oil/water emulsion or an oligonucleotide containing unmethylated CpG dinucleotide motifs and aluminium hydroxide as carrier.

3D-MPL: is a chemically detoxified form of the lipopolysaccharide (LPS) of the Gram-negative bacteria Salmonella minnesota.

Experiments performed at Smith Kline Beecham Biologicals have shown that 3D-MPL combined with various vehicles strongly enhances both the humoral immunity and a T_{H1} type of cellular immunity.

QS21: is a saponin purified from a crude extract of the bark of the Quillaja Saponaria Molina tree, which has a strong adjuvant activity: it induces both antigen-specific lymphoproliferation and CTLs to several antigens.

Experiments performed at Smith Kline Beecham Biologicals have demonstrated a clear synergistic effect of combinations of 3D-MPL and QS21 in the induction of both humoral and T_{H1} type cellular immune responses.

The oil/water emulsion is composed of 2 oils (a tocopherol and squalene), and of PBS containing Tween 80 as emulsifier. The emulsion comprises 5% squalene, 5% tocopherol, 2% Tween 80 and has an average particle size of 180 nm (see WO 95/17210).

Experiments performed at Smith Kline Beecham Biologicals have proven that the adjunction of this O/W emulsion to 3D-MPL/QS21 further increases their immunostimulant properties.

### Preparation of the Oil/Water Emulsion (2 Fold Concentrate)

Tween 80 is dissolved in phosphate buffered saline (PBS) to give a 2% solution in the PBS. To provide 100 ml two fold concentrate emulsion 5 g of DL alpha tocopherol and 5 ml of squalene are vortexed to mix thoroughly. 90 ml of PBS/Tween solution is added and mixed thoroughly. The resulting emulsion is then passed through a syringe and finally microfluidised by using an M110S Microfluidics machine. The resulting oil droplets have a size of approximately 180 nm.

### Preparation of Oil in Water Formulation.

Antigens (100 μg gp120, 20 μg NefTat, and 20 μg SIV Nef, alone or in combination) were diluted in 10 fold concentrated PBS pH 6.8 and H₂O before consecutive addition of the oil in water emulsion, 3D-MPL (50 μg), QS21 (50 μg) and 1 μg/ml thiomersal as preservative at 5 min interval. The emulsion volume is equal to 50% of the total volume (250 μl for a dose of 500 μl).

All incubations were carried out at room temperature with agitation.

CpG oligonucleotide (CpG) is a synthetic unmethylated oligonucleotide containing one or several CpG sequence motifs. CpG is a very potent inducer of T_{H1} type immunity compared to the oil in water formulation that induces mainly a mixed T_{H1}/T_{H2} response. CpG induces lower level of antibodies than the oil in water formulation and a good cell mediated immune response. CpG is expected to induce lower local reactogenicity.

Preparation of CpG oligonucleotide solution: CpG dry powder is dissolved in H₂O to give a solution of 5 mg/ml CpG.

### Preparation of CpG Formulation

The 3 antigens were dialyzed against NaCl 150 mM to eliminate the phosphate ions that inhibit the adsorption of gp120 on aluminium hydroxide.

The antigens diluted in H₂O (100 μg gp120, 20 μg NefTat and 20 μg SIV Nef) were incubated with the CpG solution (500 μg CpG) for 30 min before adsorption on Al(OH)₃ to favor a potential interaction between the His tail of NefTat and Nef antigens and the oligonucleotide (stronger immunostimulatory effect of CpG described when bound to the antigen compared to free CpG). Then were consecutively added at 5 min interval Al(OH)₃ (500 μg), 10 fold concentrated NaCl and 1 μg/ml thiomersal as preservative.

All incubations were carried out at room temperature with agitation.

### Example 14

### Immunization and SHIV Challenge Experiment in Rhesus Monkeys

### First Study

Groups of 4 rhesus monkeys were immunized intramuscularly at 0, 1 and 3 months with the following vaccine compositions:

| | | | | |
|---|---|---|---|---|
| Group 1: | Adjuvant 2 + | gp120 | | |
| Group 2: | Adjuvant 2 + | gp120 + | NefTat + | SIV Nef |
| Group 3: | Adjuvant 2 + | | NefTat* + | SIV Nef |
| Group 4 | Adjuvant 6 + | gp120 + | NefTat + | SIV Nef |
| Group 5 | Adjuvant 2 + | | NefTat + | SIV Nef |
| Group 6 | Adjuvant 2 | | | |

Adjuvant 2 comprises squalene/tocopherol/Tween 80/3D-MPL/QS21 and Adjuvant 6 comprises alum and CpG.
Tat* represents mutated Tat, in which Lys41→Ala and in RGD motif Arg78→Lys and Asp80→Glu (Virology 235: 48-64, 1997).

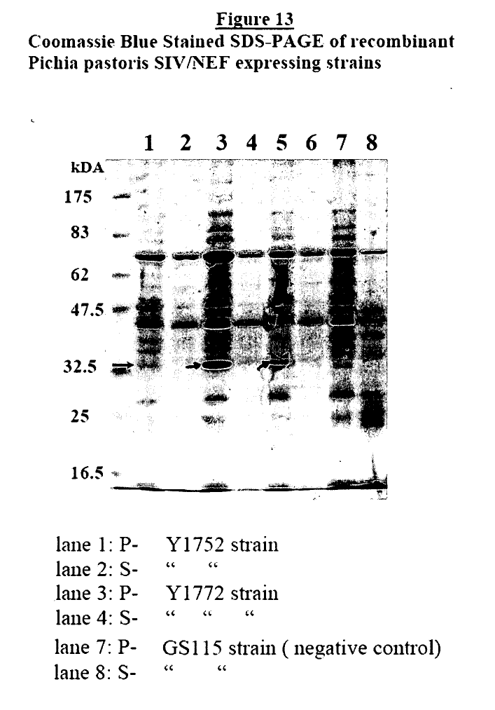

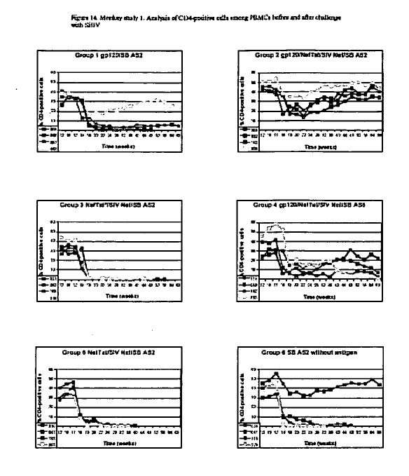

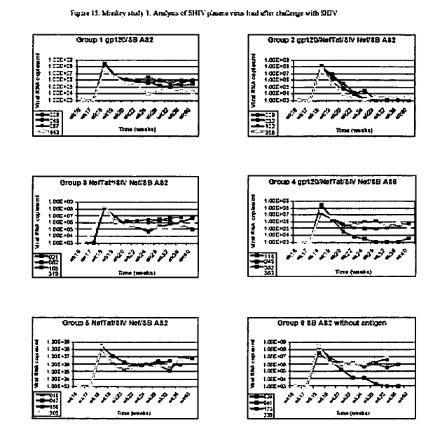

One month after the last immunization all animals were challenged with a pathogenic SHIV (strain 89.6p). From the week of challenge (wk16) blood samples were taken periodically at the indicated time points to determine the % of CD4-positive cells among peripheral blood mononuclear cells by FACS analysis (FIG. 14) and the concentration of RNA viral genomes in the plasma by bDNA assay (FIG. 15).

### Results

All animals become infected after challenge with SHIV_{89.6p}.

CD4-positive cells decline after challenge in all animals of groups 1, 3, 5 and 6 except one animal in each of groups 1 and 6 (control group). All animals in group 2 exhibit a slight decrease in CD4-positive cells and recover to baseline levels over time. A similartrend is observed in group 4 animals (FIG. 14).

Virus load data are almost the inverse of CD4 data. Virus load declines below the level of detection in ¾ group 2 animals (and in the one control animal that maintains its CD4-positive cells), and the fourth animal shows only marginal virus load. Most of the other animals maintain a high or intermediate virus load (FIG. 15).

Surprisingly, anti-Tat and anti-Nef antibody titres measured by ELISA were 2 to 3-fold higher in Group 3 (with mutated Tat) than in Group 5 (the equivalent Group with non-mutated Tat) throughout the course of the study.

At week 68 (56 weeks post challenge) all animals from the groups that had received the full antigen combination (groups 2 and 4) were still alive, while most of the animals in the other groupshad to be euthanized due to AIDS-like symptoms. The surviving animals per group were:

| | |
|---|---|
| Group 1: | 2/4 |
| Group 2: | 4/4 |
| Group 3: | 0/4 |
| Group 4 | 4/4 |
| Group 5 | 0/4 |
| Group 6 | 1/4 |

### Conclusions

The combination of gp120 and NefTat (in the presence of SIV Nef) prevents the loss of CD4-positive cells, reduces the virus load in animals infected with pathogenic SHIV_{89.6p}, and delays or prevents the development of AIDS-like disease symptoms, while gp120 or NefTat/SIV Nef alone do not protect from the pathologic consequences of the SHIV challenge.

The adjuvant 2 which is an oil in water emulsion comprising squalene, tocopherol and Tween 80, together with 3D-MPL and QS21 seems to have a stronger effect on the study endpoints than the alum/CpG adjuvant.

### Second Study

A second rhesus monkey SHIV challenge study was conducted to confirm the efficacy of the candidate vaccine gp120/NefTat+adjuvant and to compare different Tat-based antigens. The study was conducted by a different laboratory.

The design of the study was as follows.

Groups of 6 rhesus monkeys were immunized at 0, 4 and 12 weeks with injections i.m. and challenged at week 16 with a standard dose of pathogenic SHIV_{89.6p}.

Group 1 is the repeat of Group 2 in the first study.

| | | | | |
|---|---|---|---|---|
| Group 1: | Adjuvant 2 + | gp120 + | NefTat + | SIV Nef |
| Group 2: | Adjuvant 2 + | gp120 + | Tat (oxidised) | |
| Group 3: | Adjuvant 2 + | gp120 + | Tat (reduced) | |
| Group 4 | Adjuvant 2 | | | |

The follow-up/endpoints were again % CD4-positive cells, virus load by RT-PCR, morbidity and mortality

### Results

All animals except one in group 2 become infected after challenge with SHIV_{89.6p}.

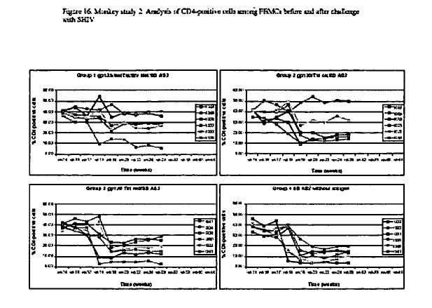

CD4-positive cells decline significantly after challenge in all animals of control group 4 and group 3, and in all but one animals of group 2. Only one animal in group 1 shows a marked decrease in CD4-positive cells. Unlike the animals from the first study, the monkeys in the second experiment display a stabilisation of CD4-positive cells at different levels one month after virus challenge (FIG. 16). The stabilisation is generally lower than the initial % of CD4-positive cells, but will never lead to a complete loss of the cells. This may be indicative of a lower susceptibility to SHIV-induced disease in the monkey population that was used for the second study. Nonetheless, a beneficial effect of the gp120/NefTat/SIV Nef vaccine and the two gp120/Tat vaccines is demonstrable. The number of animals with a % of CD4-positive cells above 20 is 5 for the vaccinated animals, while none of the control animals from the adjuvant group remains above that level.

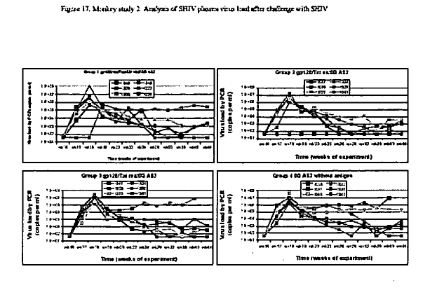

Analysis of RNA plasma virus loads confirms the relatively low susceptibility of the study animals (FIG. 17). Only 2 of the 6 control animals maintain a high virus load, while the virus disappears from the plasma in the other animals. Thus, a vaccine effect is difficult to demonstrate for the virus load parameter.

### Conclusions

Analysis of CD4-positive cells indicates that the vaccine gp120/NefTat+adjuvant (in the presence of SIV Nef) prevents the drop of CD4-positive cells in most vaccinated animals This is a confirmation of the result obtained in the first SHIV study. Due to the lack of susceptibility of the study animals, the virus load parameter could not be used to demonstrate a vaccine effect. Taken together, the combination of gp120 and Tat and Nef HIV antigens provides protection against the pathologic consequences of HIV infection, as evidenced in a SHIV model.

The Tat alone antigens in combination with gp120 also provide some protection from the decline of CD4-positive cells. The effect is less pronounced than with the gp120/NefTat/SIV Nef antigen combination, but it demonstrates that gp120 and Tat are able to mediate some protective efficacy against SHIV-induced disease manifestations.

The second SHIV challenge study was performed with rhesus monkeys from a source completely unrelated to the source of animals from the first study. Both parameters, % of CD4-positive cells and plasma virus load, suggest that the animals in the second study were less susceptible to SHIV-induced disease, and that there was considerably greater variability among the animals. Nonetheless, a beneficial effect on the maintenance of CD4-positive cells of the gp120/NefTat/SIV Nef vaccine was seen with the experimental vaccine containing gp120/NefTat and SIV Nef. This indicates that the vaccine effect was not only repeated in a separate study, but furthermore demonstrated in an unrelated monkey population.

1.-19. (canceled)

20. A method of prophylactically or therapeutically immunizing a human against HIV infection, which method comprises administering to said human in need thereof an effective amount of an immunogenic formulation comprising an HIV Nef protein or polynucleotide and an HIV gp120 protein or polynucleotide, wherein the Nef interaction with gp120 produces a synergistic effect.

21. The method of claim 20, wherein administering the immunogenic formulation reduces the HIV viral load in an HIV infected human.

22. The method of claim 20, wherein administering the immunogenic formulation results in a maintenance of CD4+ T cell levels over those found in the absence of administration.

23. The method of claim 20, wherein the immunogenic formulation further comprises an antigen selected from the group of: tat, gag, rev, vif, vpr, and vpu.

24. The method of claim 20, wherein the Nef protein is reduced.

25. The method of claim 20, wherein the Nef protein is carbamidomethylated.

26. The method of claim 20, wherein the Nef protein is oxidised.

27. The method of claim 20, wherein the immunogenic composition comprises an adjuvant.

28. The method of claim 27, wherein the adjuvant is a TH1 inducing adjuvant.

29. The method of claim 28, wherein the adjuvant comprises a monophosphoryl lipid.

30. The method of claim 29, wherein the monophosphoryl lipid is selected from the group of: monophosphoryl lipid A, 3-de-O-acylated monophsphoryl lipid A, and monophosphoryl lipid A derivatives thereof.

31. The method of claim 28, wherein the adjuvant comprises an oligonucleotide comprising an unmethylated CpG.

32. The method of claim 31, further comprising an aluminium salt.

33. The method of claim 27, wherein the adjuvant comprises a saponin.

34. The method of claim 20, wherein the immunogenic formulation comprises an oil in water emulsion.

35. A method of prophylactically or therapeutically immunizing a human against HIV infection, which method comprises administering to said human an effective amount of a an immunogenic formulation suitable for a prime-boost delivery wherein the immunogenic formulation comprises an HIV Nef protein or polynucleotide and an HIV gp120 protein or polynucleotide, wherein the Nef interaction with gp120 produces a synergistic effect.

36. An immunogenic formulation comprising an HIV Nef protein or polynucleotide in combination with an HIV gp120 protein or polynucleotide, wherein the Nef interaction with gp120 produces a synergistic effect.
